(19) United States
(12) Patent Application Publication
(10) Pub. No.: US 2012/0115964 A1
(43) Pub. Date: May 10, 2012

(54) XANTHAN GUM AND SWELLABLE PARTICULATE CONTAINING COMPOSITION AND USES THEREOF

(75) Inventors: Timothy John Foster, Nottingham (GB); John Richard Mitchell, Nottingham (GB); Mitaben Dhirajlal Lad, Nottingham (GB)

(73) Assignee: THE UNIVERSITY OF NOTTINGHAM, Nottingham, Nottinghamshire (GB)

(21) Appl. No.: 13/265,291

(22) PCT Filed: Apr. 20, 2010
(86) PCT No.: PCT/GB2010/050642

§ 371 Date: Jan. 30, 2012

(30) Foreign Application Priority Data
Apr. 20, 2009 (GB) 0906771.1

(51) Int. Cl. A61K 47/36; A23L 1/054; A23L 1/30; A23L 1/0534; A23L 1/0528; A61P 3/04; A23L 1/0522

(52) U.S. Cl. 514/777; 426/654; 426/648

(57) ABSTRACT

This invention relates to a composition comprising at least about 0.2% w/w xanthan gum and at least about 6% w/w of a swellable particulate, and to uses thereof.

The present invention relates to a composition comprising xanthan gum and a swellable particulate, and to uses of such a composition, for example, as a thickening agent. The invention also relates to the use of xanthan gum with a material containing a swellable particulate to cause an increase in viscosity.

In particular the present invention relates to a novel and inventive way of decreasing the viscosity of a composite system below that which might be expected given the component ingredients, and a system that upon dilution either maintains or increases its viscosity. The solution provided by the invention is a mixture of xanthan gum (a bacterial exudate gum) and a swellable particulate, such as a non-polymeric polyelectrolyte viscosifying agent e.g. swellable starch, which when admixed show a decrease in viscosity when compared to what might be expected from the viscosities of the two starting components alone. In some embodiments, addition of further solvent may cause the viscosity to be maintained or increased, where a decrease in viscosity might be expected.

Food systems often contain mixtures of xanthan gum and starch. Typical products would be dressings and sauces, in which, for reasons of texture quality control, the xanthan gum and starch are used during a cooking process in order to gelatinise the starch in the presence of the xanthan gum. Many researchers have studied this phenomenon, studying different water soluble polymeric gums, and starches from different sources and with different chemical/physical modifications. Recent developments have investigated the structuring of these product types under fast, late stage/distributed manufacture production procedures, in which the water is added to concentrated mixes and the polysaccharides then undergo competitive hydration for the available water.

In the present invention the desired effect has been found by using swellable particulates, such as swellable starch granules, swellable natural fibres (e.g. citrus fibre), swellable food systems (e.g. porridge) and swellable gel particles, with xanthan gum. The market of late stage customisation/low water usage is potentially huge and as such the technology of the present invention may be applicable there. Additionally, the present invention provides a product that continues to viscosify upon dilution, for example in the stomach, this may be used in nutritional/nutraceutical supplements for the control of the growing obesity problem.

According to a first aspect, the invention provides a composition comprising at least about 0.2% w/w xanthan gum and at least about 6% w/w of a swellable particulate.

A swellable particulate refers to a particulate that swells at least in water, but typically in other liquids or solutions.

Preferably the swellable particulate is readily dispersible in water. Preferably the swellable particulate is readily dispersible in other potable liquid or foodstuff, such as orange juice or milk.

Preferably before dilution the particulates are discrete and free flowing particulates. Alternatively, rather than free flowing, the particulates may be mixed with oil or lecithin to form a liquid dispersion prior to further dilution. Preferably once diluted, for example in water or another liquid, the particulates remain as discrete entities suspended within the liquid. The swellable particulate may be swellable in a cold and/or a warm solution. Preferably the swellable particulate is swellable in a solution at a temperature below about 60° C., more preferably the swellable particulate is swellable in a solution at room temperature. Room temperature may be about 25° C. Preferably the swellable particulate may be referred to as a cold swelling particulate.

The swellable particulate may comprise polymer molecules that have been stabilized in the particulate form by either chemical or physical crosslinking. Alternatively, or additionally, the swellable particulate may comprise a starch polymer which has been pre-gelatinsed, which when dispersed in cold water produces swollen particles. The polymer molecules may be charged or uncharged. The polymer may be starch.

The swellable particulates preferably have a mass median particulate size of between about 1 μm and about 2mm, preferably between about 1 μm and about 500 μm, preferably between about 1 μm and about 50 μm.

The swellable particulates may be of any suitable porosity or density.

The particulate matter may be a particle produced by the method of WO2006/065136 which describes the production of swellable particulates of xanthan gum and/or starch.

Preferably, in a composition of the invention, the swellable particulate swells to compete for water with polymeric, non particulate, xanthan gum.

Preferably the at least about 0.2% xanthan gum is composed of a xanthan gum which forms a polymeric solution in water, preferably the polymeric solution is formed upon cold mixing. Preferably the xanthan gum is not in the particulate form once dispersed in cold water.

Preferably the swellable particulate would form a sediment layer when diluted, this in contrast to a polymeric solution that would form a more dilute solution when diluted.

In the composition of the invention the xanthan gum may be driven/trapped in a highly concentrated anisotropic solution wherein the xanthan gum is hydrated into a concentrated liquid crystalline phase.

The swellable particulate may comprise one or more of the following: starch, modified starch, citrus fibres, particulate xanthan gum (such as hydraxan), fibrous cellulose (such as nata de coco), oats, swellable gel particles e.g. dried fluid gel particulates created as described in Norton, Jarvis and Foster, International Journal of Biological Macromolecules (1999), 26, 255-261, or particulates described in WO9512988-A and surfactant micelles. Where the swellable particulate comprises starch the starch may be derived from potato, maize, tapioca, rice, wheat, cassava, pea or any other suitable material. The starch may be physically or chemically modified. If maize starch is used the maize starch may be a modified waxy maize starch. Preferably if starch is used it is a starch capable of being hydrated at below 60° C. Preferably the starch is not a cook-up starch.

Preferably the composition comprises between about 6% w/w and about 25% w/w of a swellable particulate. Preferably the composition comprises at least about 10% w/w of a swellable particulate. Preferably the composition comprises between about 10% w/w and about 25% w/w of a swellable particulate.

Preferably the composition comprises between about 0.2% w/w and about 4% w/w xanthan gum. Preferably the composition comprises less than about 10% xanthan gum.

The composition may further comprise at least about 5% w/w oil, and/or at least about 1% w/w lecithin. The addition of oil or lecithin may help to hydrate the xanthan gum and/or the swellable particulate. Preferably if oil and/or lecithin are used the xanthan gum and/or the swellable particulate are dispersed in oil and/or lecithin before dilution to the final concentration.

The oil may be any edible oil which is liquid at room temperature. The oil may be one or more of sun flower oil, olive oil, soybean oil, corn oil, cottonseed oil, nut oil, rapeseed oil and low melting butterfat fractions, or mixtures thereof.

The composition may further comprise salt. The salt may be one or more of sodium chloride, potassium chloride, potassium sorbate, sodium fluoride, potassium fluoride, sodium iodide and potassium iodide or any other suitable salt. Preferably the salt is not a divalent or a trivalent salt. The concentration of salt may range from about 0.01M to about 1M, or higher.

Preferably the composition further comprises water, Preferably at least about 50%, at least about 55%, at least about 60%, at least about 65%, at least about 70%, at least about 75%, at least about 80% of the composition is water.

The composition may further comprise one or more of the following, a flavouring, a colouring, a preservative and a vitamin.

The composition may be provided as a paste. The paste may have a viscosity in the range of about 600 to about 12000 cP, more preferably in the range about 1800 to about 4000 cP, even more preferably about 2000 to about 3500 cP.

Preferably a composition according to the invention has a viscosity lower than expected, that is, preferably the viscosity of the composition is less than the viscosity that would be observed in a composition containing only the swellable particulate. This is in contrast to a composition containing a swellable particulate and a different hydrocolloid, for example guar gum or alginate, where the viscosity of the composition would be the same or greater than a composition containing only the swellable particulate.

In a preferred embodiment of the invention, the composition comprises at least about 0.2% w/w xanthan gum and at least about 6% w/w, and preferably at least about 10%, of a modified waxy maize starch.

In a further preferred embodiment of the invention, the composition comprises at least about 0.2% w/w xanthan gum and at least about 6% w/w, and preferably at least about 10%, of a swellable particulate of xanthan gum, such as hydraxan.

Preferably, and surprisingly, the composition of the invention may show an increase or maintenance in viscosity upon dilution. That is, when the composition of the invention is diluted the viscosity of the resulting composition may be greater than, or the same as, the viscosity of the composition according to the invention before dilution. In order to observe an increase or maintenance in viscosity the dilution may be between about 1 part composition according to the invention and about 1 part, preferably about 0.5 part, to about 0.1 part liquid, for example water, milk, juice or gastric fluids. Preferably this increase in viscosity upon dilution is observed in compositions wherein the swellable particulate is a modified waxy maize starch. Alternatively the swellable particulate may be particulate xanthan gum.

Preferably where an increase in viscosity is observed upon dilution the composition prior to dilution comprises at least about 0.2% w/w xanthan gum and at least about 6% w/w of a modified waxy maize starch. More preferably, the composition comprises, between about 0.2% and about 4% w/w xanthan gum and about 6% to about 20% w/w of a modified waxy maize starch. Preferably the dilution is about 1 part composition according to the invention to about 0.5 part liquid.

Preferably at higher dilutions, say about 1 part composition according to the invention to about 8 to about 16 parts liquid, the composition will serve to thicken the liquid, and the resulting solution will have a viscosity less than the viscosity of the composition according to the invention before dilution, and greater than the liquid before addition of the composition according to the invention.

According to a further aspect, the invention provides a composition according to the invention for use as a thickening agent, in particular in for use in foodstuffs.

According to a yet further aspect, the invention provides the use of a composition according to the invention as a thickening agent, in particular in foodstuffs. More specifically, a composition according to the invention may be added to a foodstuff in order to increase the viscosity of the foodstuff.

The term thickening agent is intended to refer to any substance/composition which when added to another substance/composition causes the another substance/composition to increase in viscosity. The skilled man will be readily able to determine by simple trial and error how much of a particular thickening agent is required to observe a particular increase in viscosity.

According to another aspect, the invention provides a foodstuff which has been thickened using a composition according to the invention.

According to yet another aspect, the invention provides a foodstuff comprising a composition according to the invention that upon dilution with a liquid, such as water, will increase in viscosity. The foodstuff may consist only of a composition according to the invention. Preferably a dilution of between about 1 part composition and about 1 part, preferably about 0.5 part, and about 0.1 part liquid/diluent would be used.

The foodstuff may be used to give an individual a feeling of satiety. This may be achieved by ingestion of a foodstuff/composition according to the invention which once in the individual's stomach is diluted by gastric fluids which cause an increase in the viscosity of the foodstuff/composition and hence give a feeling of satiety to the individual.

According to yet another aspect, the invention provides a process for using the composition of the invention as a thickening agent in foodstuffs comprising the step of adding the composition to a foodstuff.

The food or foodstuff in any aspect of the invention may be a sauce, spread, soup, gravy, dessert, filling, batter, dough, cereal, such as porridge oats, a drink or any other edible product.

According to a still further aspect, the invention provides use of xanthan gum as a thickening agent wherein the xanthan gum is added, or intended to be added, to a foodstuff containing at least about 6% w/w, preferably at least about 10% w/w, of a swellable particulate, and wherein when the xanthan gum is added to give a concentration of at least about 0.2% w/w xanthan. Preferably the concentration of swellable particulate is between about 6% w/w and about 25% w/w. Preferably the concentration of xanthan gum is between about 0.2% w/w and about 10% w/w. The foodstuff may be a cereal, for example, porridge oats.

According to another aspect, the invention provides a nutritional or nutraceutical composition comprising the composition of the invention. The nutritional or nutraceutical composition may be for use as a dietary aid, or for the treatment of obesity, this may be achieved by using a composition as described above to increase satiety in an individual.

This aspect of the invention is particularly applicable to the composition of the invention which is able to increase or maintain viscosity upon dilution, this would, for example, allow a product to continue to viscosify upon dilution in the stomach and thus lead to a feeling of greater satiety.

According to a further aspect, the invention provides a pharmaceutical composition comprising a composition according to the invention and a pharmaceutically acceptable carrier or excipient. The pharmaceutical may be use in the treatment of obesity and/or for the treatment of dysphagia.

A pharmaceutical, nutraceutical or nutritional composition according to the invention may be administered to a dysphagic patient in order to invoke a swallow response.

According to a still further aspect, the invention provides a further composition comprising between about 0.8 and about 60.6% xanthan gum, about 28.6 and about 96.6% of a swellable particulate and about 1.6 and about 44.6% oil or lecithin. Preferably the composition of this aspect of the invention consists only of xanthan gum, swellable particulate and oil/lecithin.

It will be appreciated that, where appropriate, all optional or preferable features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

Embodiments and examples of the present invention will now be described herein, by way of example only, with reference to the following figures.

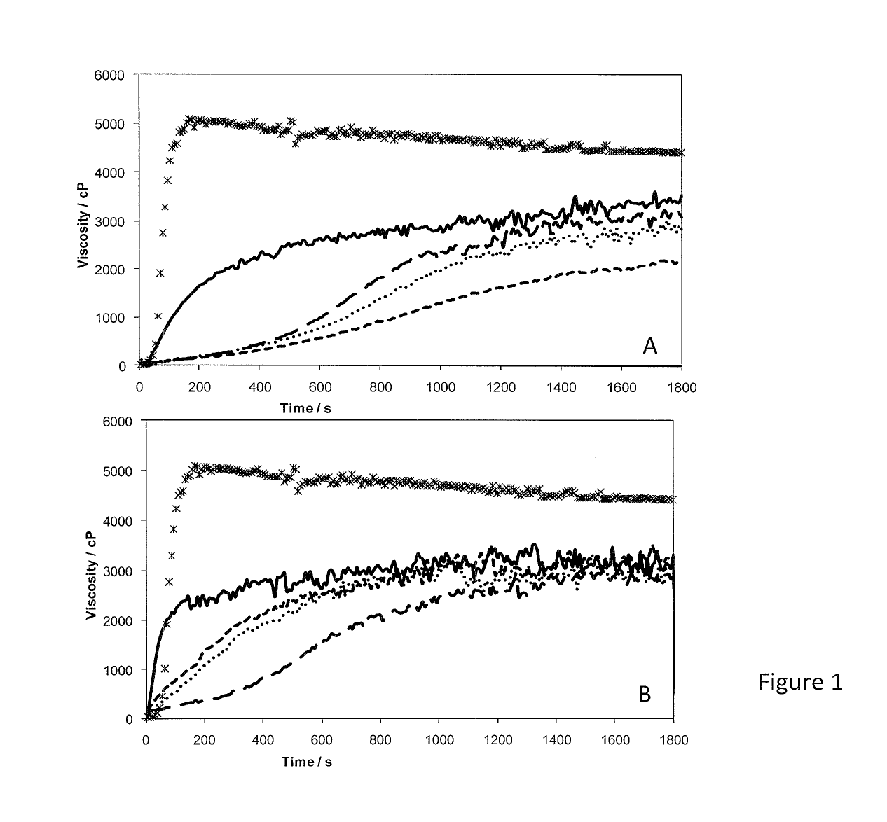

FIG. 1 illustrates the change in viscosity in a Rapid Visco Analyser for a mixture of 10% starch (UT2), 5% oil (***) (the upper line in both FIGS. 1A and 1B)+xanthan gum (sigma) (••••), xanthan gum (supra) ( [symbol] - solid line), xanthan gum (200) ( [symbol] [symbol] ) and xanthan gum (80) (----). The amount of xanthan gum varies between figures A and B, 0.5% and 2% respectively.

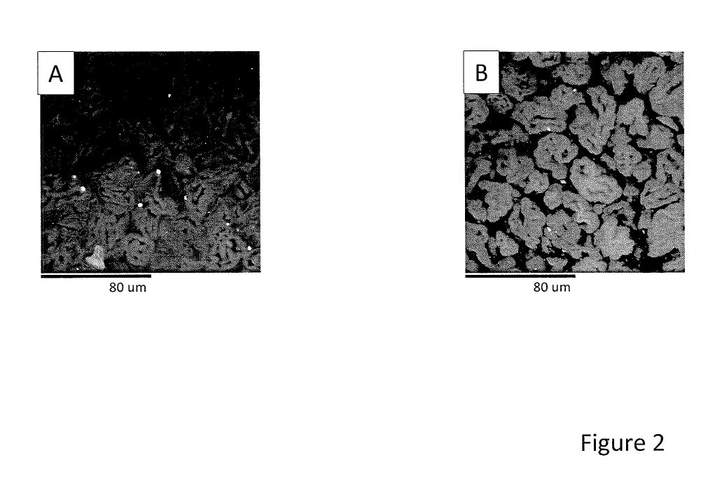

FIG. 2 shows confocal micrographs of 10% UT2 starch alone (A) and 10% starch (UT2)+2% xanthan gum (B).

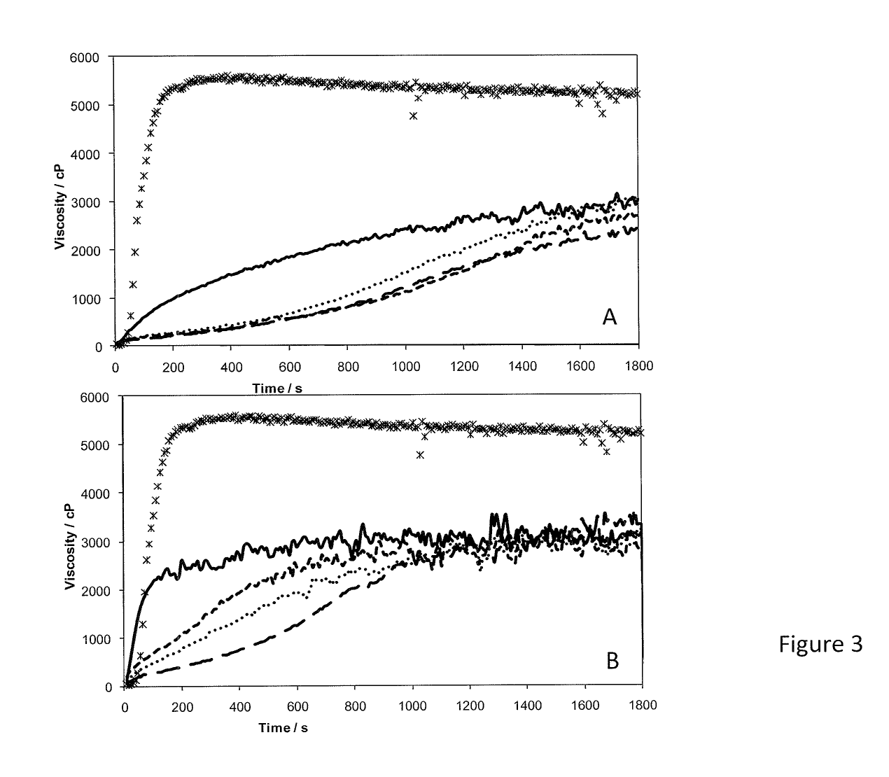

FIG. 3 illustrates the change in viscosity in a Rapid Visco Analyser for a mixture of 10% starch (UT4), 5% oil (***)+xanthan gum (sigma) (••••), xanthan gum (supra) ( [symbol] ), xanthan gum (200) ( [symbol] [symbol] ) and xanthan (80) (----). The amount of xanthan gum varies between figures A and B, 0.5% and 2% respectively.

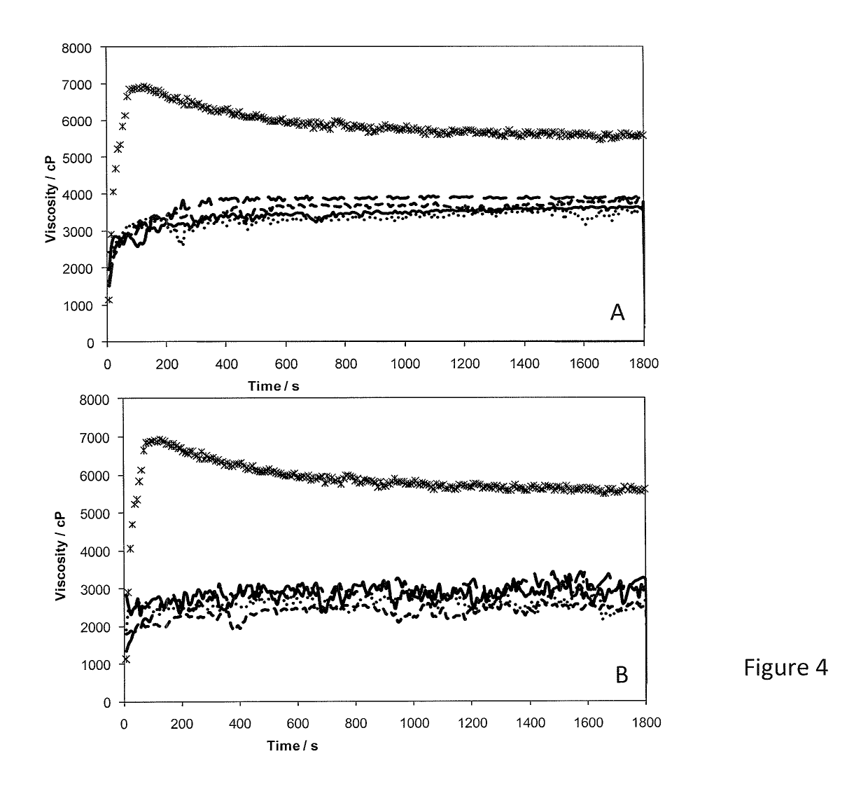

FIG. 4 illustrates the change in viscosity in a Rapid Visco Analyser for a mixture of 10% potato starch, 5% oil (***)+xanthan gum (sigma) (••••), xanthan gum (supra) ( [symbol] ), xanthan gum (200) ( [symbol] [symbol] ) and xanthan gum (80) (----). The amount of xanthan gum varies between figures A and B, 0.5% and 2% respectively.

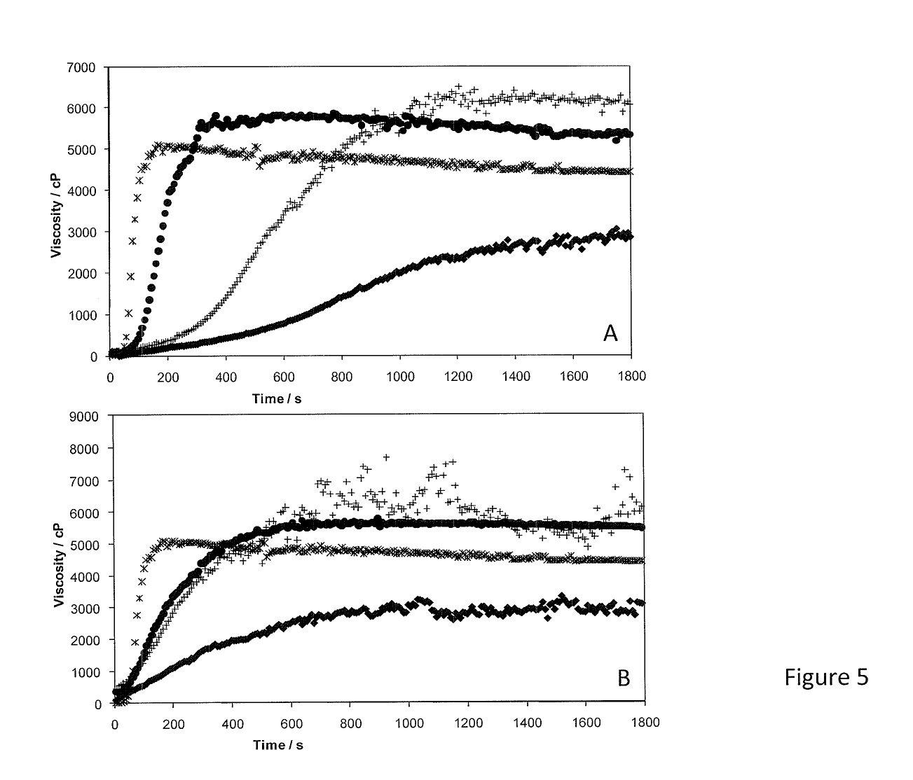

FIG. 5 illustrates the change in viscosity in a Rapid Visco Analyser for a mixture of 10% starch (UT2), 5% oil (***)+xanthan gum (sigma) (♦), guar gum () and alginate (+).The amount of hydrocolloid varies between figures A and B, 0.5% and 2% respectively.

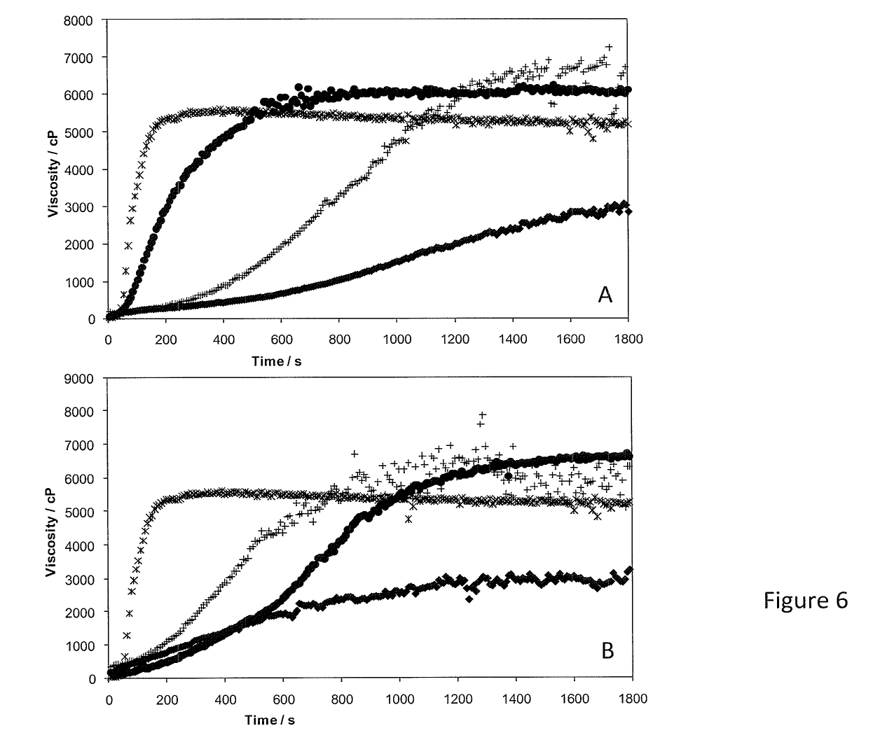

FIG. 6 illustrates the change in viscosity in a Rapid Visco Analyser for a mixture of 10% starch (UT4), 5% oil (*)+xanthan gum (sigma) (♦), guar gum () and alginate (+).The amount of hydrocolloid varies between figures A and B, 0.5% and 2% respectively.

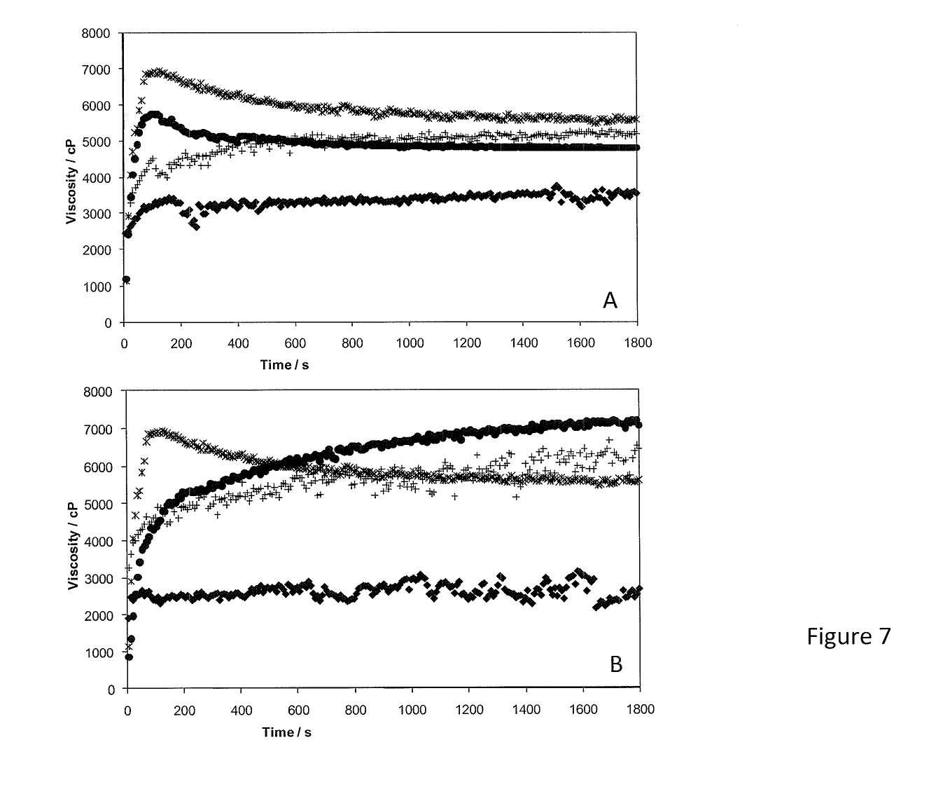

FIG. 7 illustrates the change in viscosity in a Rapid Visco Analyser for a mixture of 10% potato starch, 5% oil (*)+xanthan gum (sigma) (♦), guar gum () and alginate (+). The amount of hydrocolloid varies between figures A and B, 0.5% and 2% respectively.

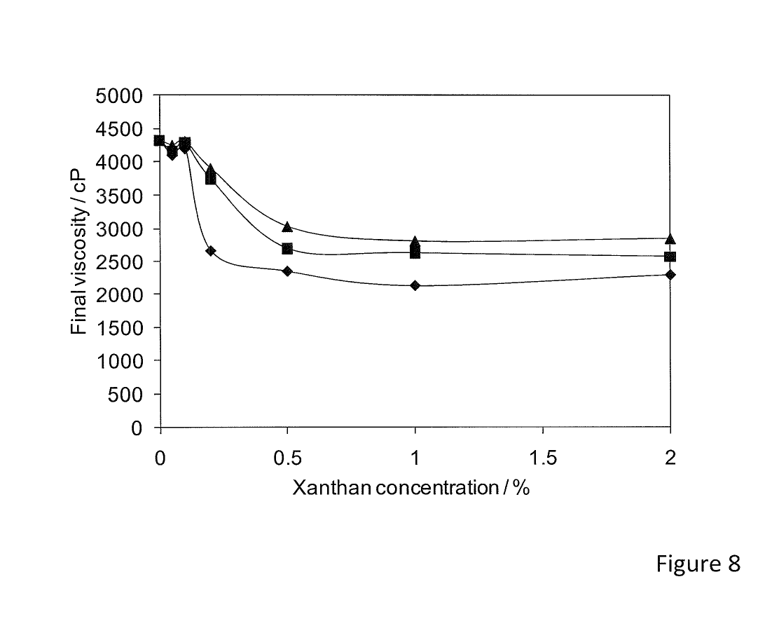

FIG. 8 illustrates the final viscosity profiles (after 30 mins of mixing in the Rapid Visco Analyser) as a function of increase varying types of xanthan gum concentration sigma (♦) supra (▪) and 200 (▴) with constant starch (UT2) concentration (10%).

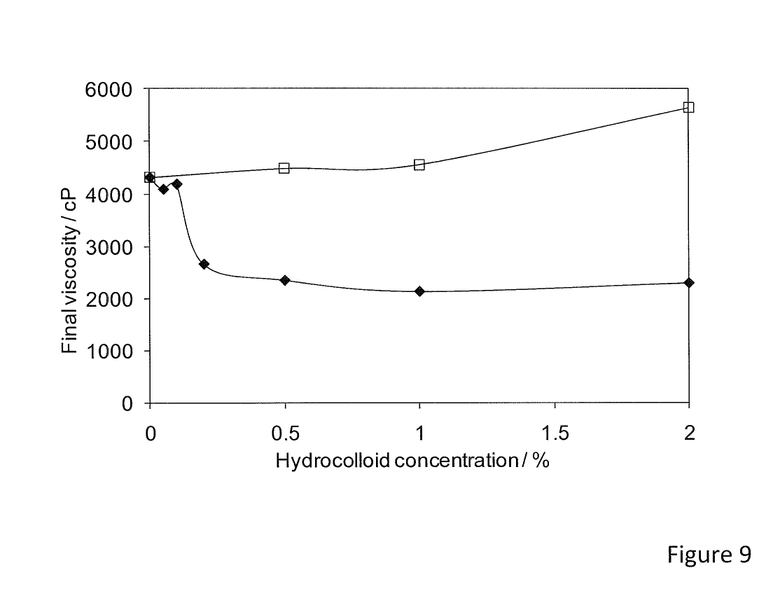

FIG. 9 illustrates the final viscosity profiles (after 30 mins of mixing in the Rapid Visco Analyser) as a function of increase in xanthan gum concentration sigma (♦) and guar gum (□) with constant starch (UT2) concentration (10%).

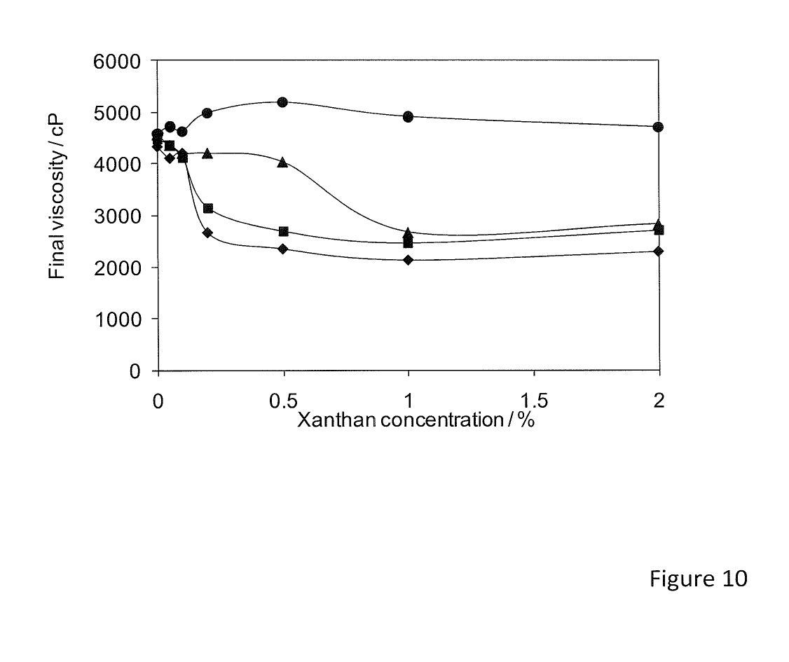

FIG. 10 illustrates the final viscosity profiles (after 30 mins of mixing in the Rapid Visco Analyser) as a function of increase salt concentration 0 M (♦), 0.01M (▪), 0.1 M (▴) and 1 M () with a constant starch (UT2) concentration (10%) and varying xanthan gum (sigma) concentration.

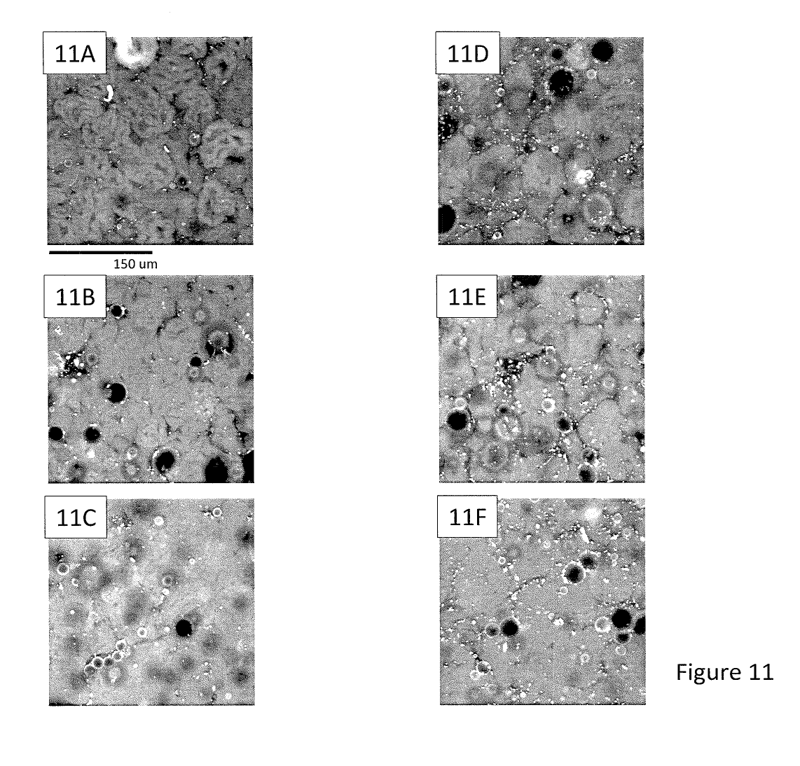

FIG. 11 shows confocal micrographs of 10% starch (UT2)+0.5% xanthan gum (sigma) 0 M salt (11A), 0.1 M salt (11B) and 1 M salt (11C). 10% starch (UT2)+2% xanthan gum (sigma) 0 M salt (11D), 0.1 M salt (11E) and 1 M salt (11F).

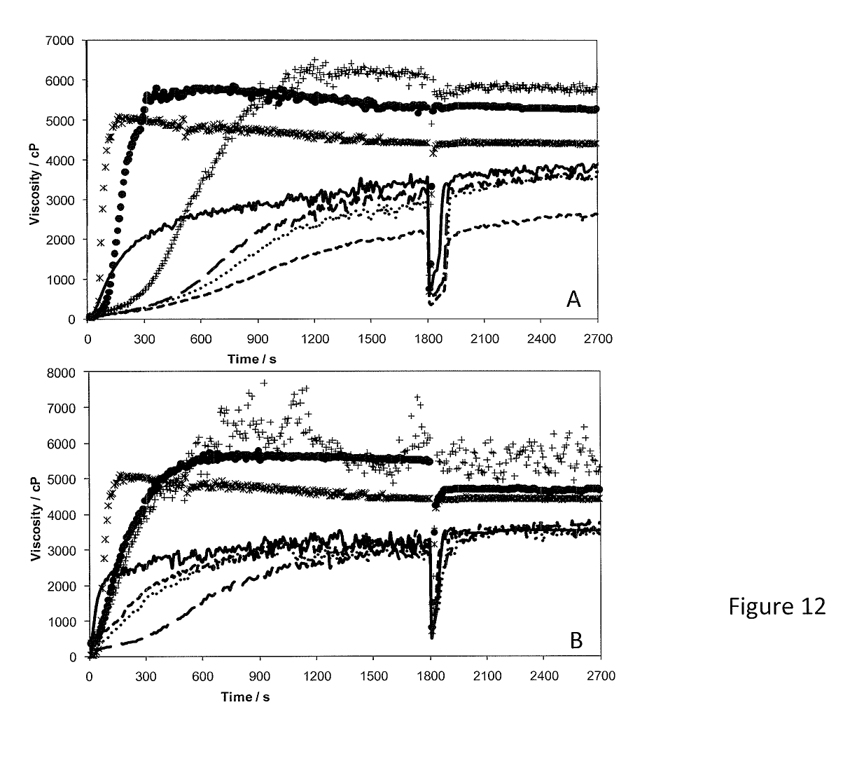

FIG. 12 illustrates the change in viscosity in a Rapid Visco Analyser before (made with water) and after 10% dilution (with water) for a mixture of 10% starch (UT2), 5% oil (***)+xanthan gum (sigma) (••••), xanthan gum (supra) ( [symbol] ), xanthan gum (200) ( [symbol] [symbol] ), xanthan gum (80) (----) guar gum () and alginate (+ + +). The amount of hydrocolloid varies between figures A and B, 0.5% and 2% respectively.

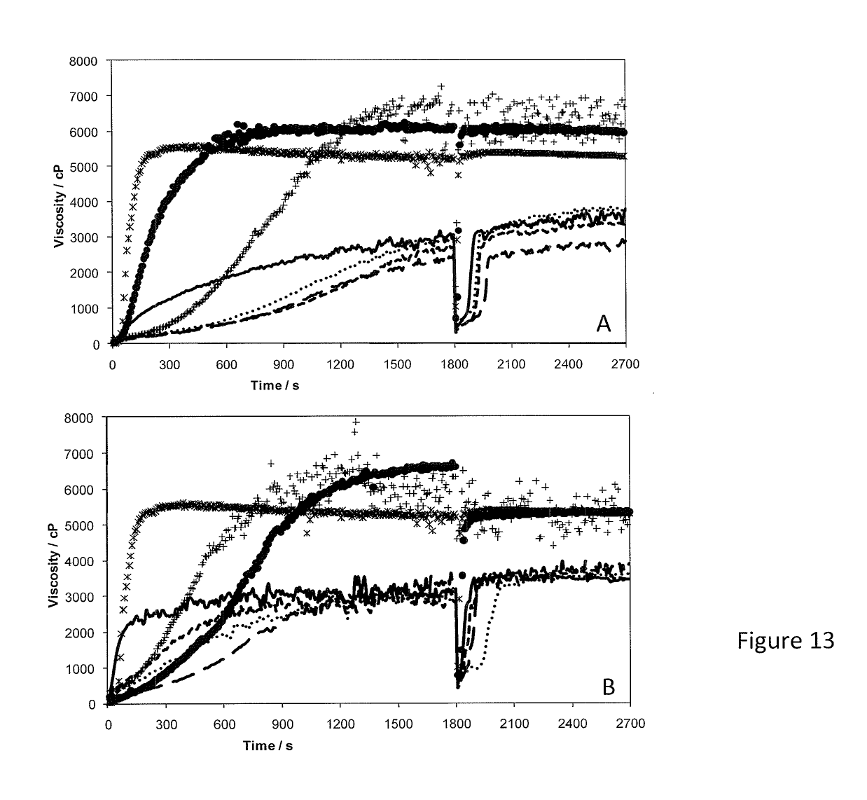

FIG. 13 illustrates the change in viscosity in a Rapid Visco Analyser before (made with water) and after 10% dilution (with water) for a mixture of 10% starch (UT4), 5% oil (***)+xanthan gum (sigma) (••••), xanthan gum (supra) ( [symbol] ) xanthan gum (200) ( [symbol] [symbol] ), xanthan gum (80) (----) guar gum () and alginate (+ + +). The amount of hydrocolloid varies between figures A and B, 0.5% and 2% respectively.

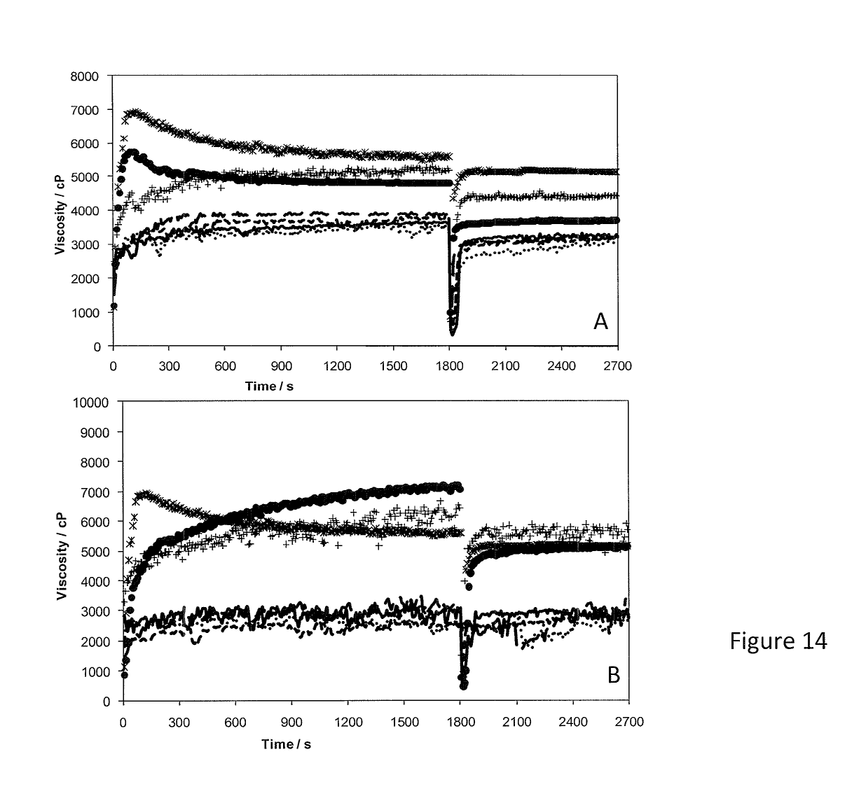

FIG. 14 illustrates the change in viscosity in a Rapid Visco Analyser before (made with water) and after 10% dilution (with water) for a mixture of 10% potato starch, 5% oil (***)+xanthan gum (sigma) (••••), xanthan gum (supra) ( [symbol] ), xanthan gum (200) ( [symbol] [symbol] ), xanthan gum (80) (----) guar gum () and alginate (+ + +). The amount of hydrocolloid varies between figures A and B, 0.5% and 2% respectively.

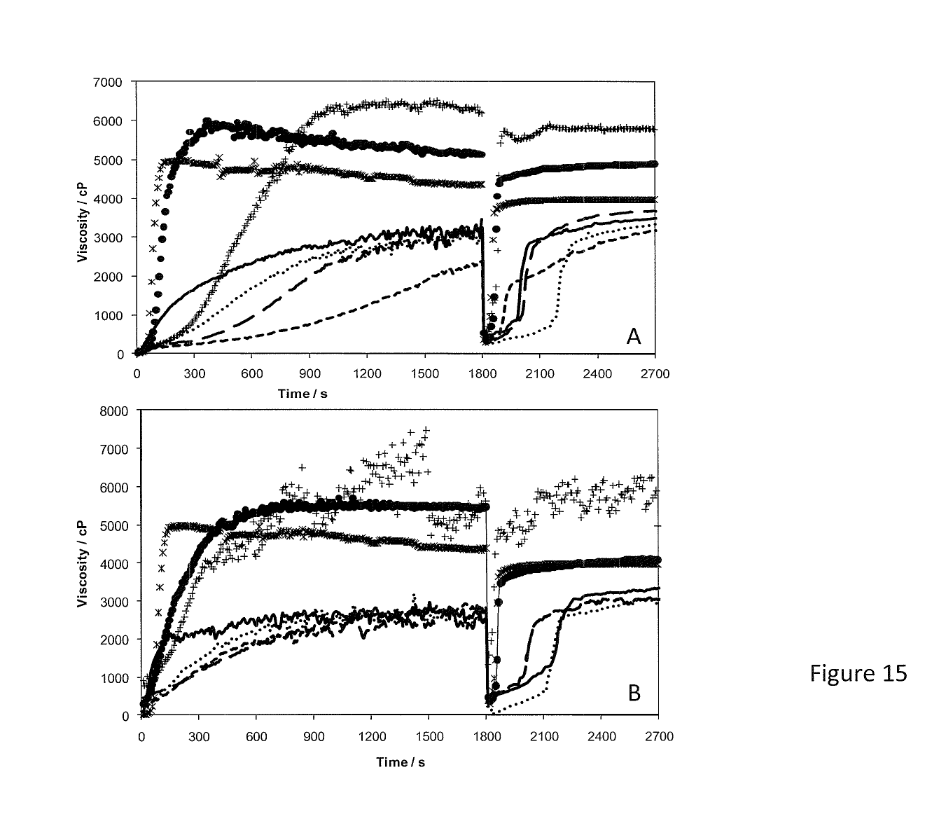

FIG. 15 illustrates the change in viscosity in a Rapid Visco Analyser before (made with water) and after 20% dilution (with water) for a mixture of 10% starch (UT2), 5% oil (***)+xanthan gum (sigma) (••••), xanthan gum (supra) ( [symbol] ), xanthan gum (200) ( [symbol] [symbol] ), xanthan gum (80) (----) guar gum () and alginate (+ + +). The amount of hydrocolloid varies between figures A and B, 0.5% and 2% respectively.

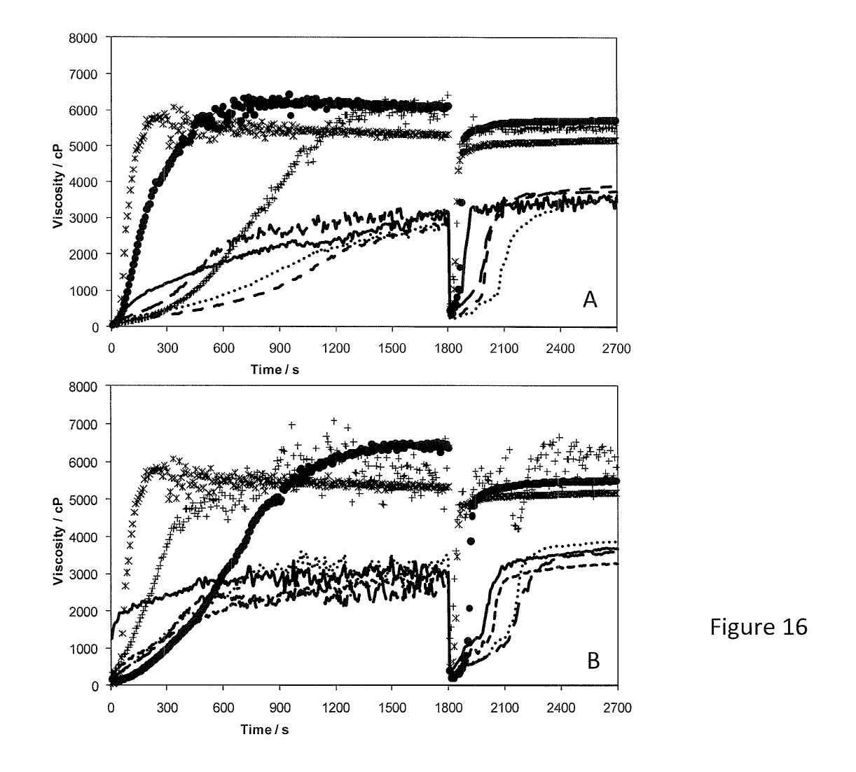

FIG. 16 illustrates the change in viscosity in a Rapid Visco Analyser before (made with water) and after 20% dilution (with water) for a mixture of 10% starch (UT4), 5% oil (***)+xanthan gum (sigma) (••••), xanthan gum (supra) ( [symbol] ), xanthan gum (200) ( [symbol] [symbol] ), xanthan gum (80) (----) guar gum () and alginate (+ + +). The amount of hydrocolloid varies between figures A and B, 0.5% and 2% respectively.

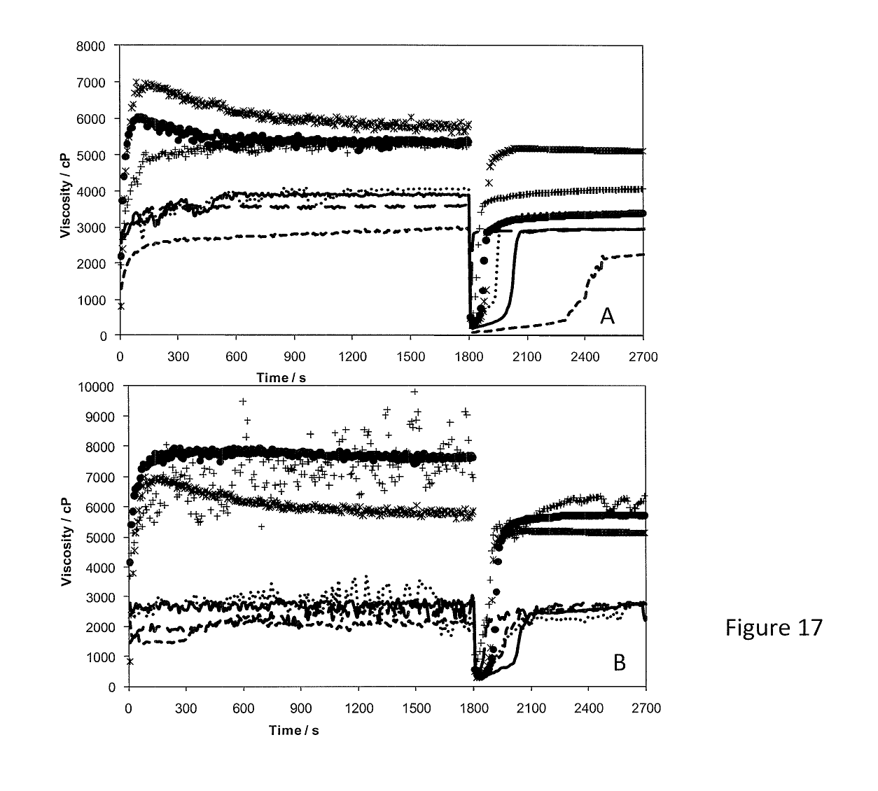

FIG. 17 illustrates the change in viscosity in a Rapid Visco Analyser before (made with water) and after 20% dilution (with water) for a mixture of 10% potato starch, 5% oil (***)+xanthan gum (sigma) (••••), xanthan gum (supra) ( [symbol] ), xanthan gum (200) ( [symbol] [symbol] ), xanthan gum (80) (----) guar gum () and alginate (+ + +). The amount of hydrocolloid varies between figures A and B, 0.5% and 2% respectively.

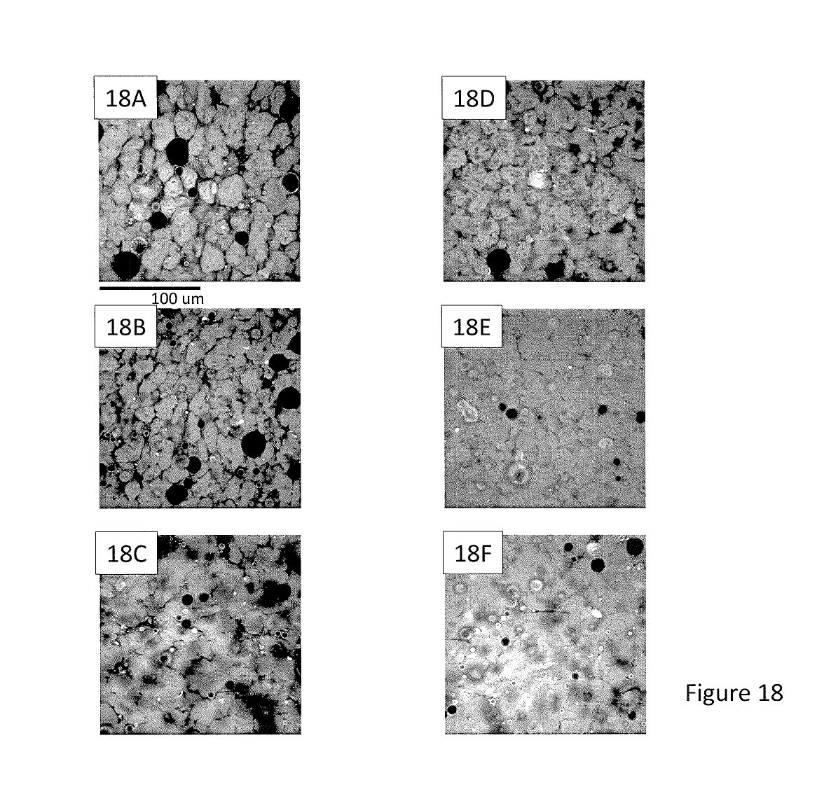

FIG. 18 shows confocal micrographs of 10% starch (UT2)+2% xanthan gum (supra) 0 M salt (18A), 0.1 M salt (18B) and 1 M salt (18C). 10% starch (UT2)+0.5% xanthan gum (supra) 0 M salt (18D), 0.1 M salt (18E) and 1 M salt (18F).

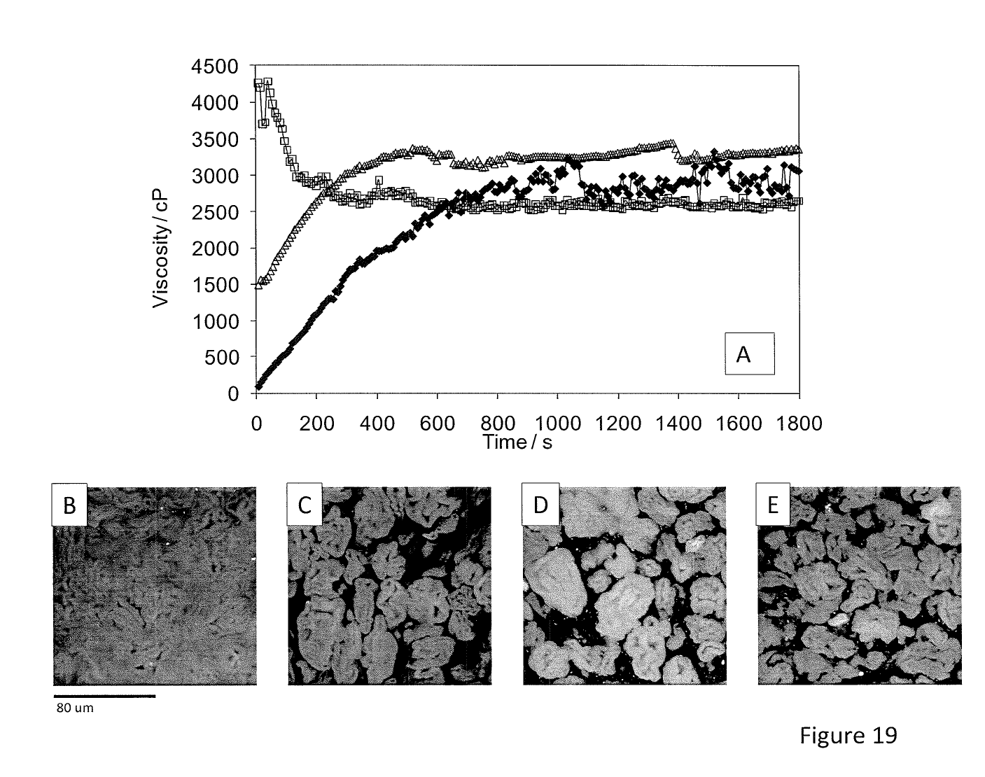

FIG. 19A illustrates the change in viscosity in a Rapid Visco Analyser (A) for a mixture of 10% starch (UT2)+5% oil+2% xanthan gum (instant mix) (♦), 10% starch (UT2) powder to 2% xanthan gum paste (Δ) and 2% xanthan gum powder added to 10% starch (UT2) paste (□). FIGS. 19A, B, C, D and E shows confocal micrographs of 10% starch (UT2) alone (B), 10% starch (UT2) paste+2% xanthan gum addition as powder (C), 10% starch (UT2) powder+2% xanthan gum paste (D) and 10% starch (UT2)+2% xanthan gum instant mix (E).

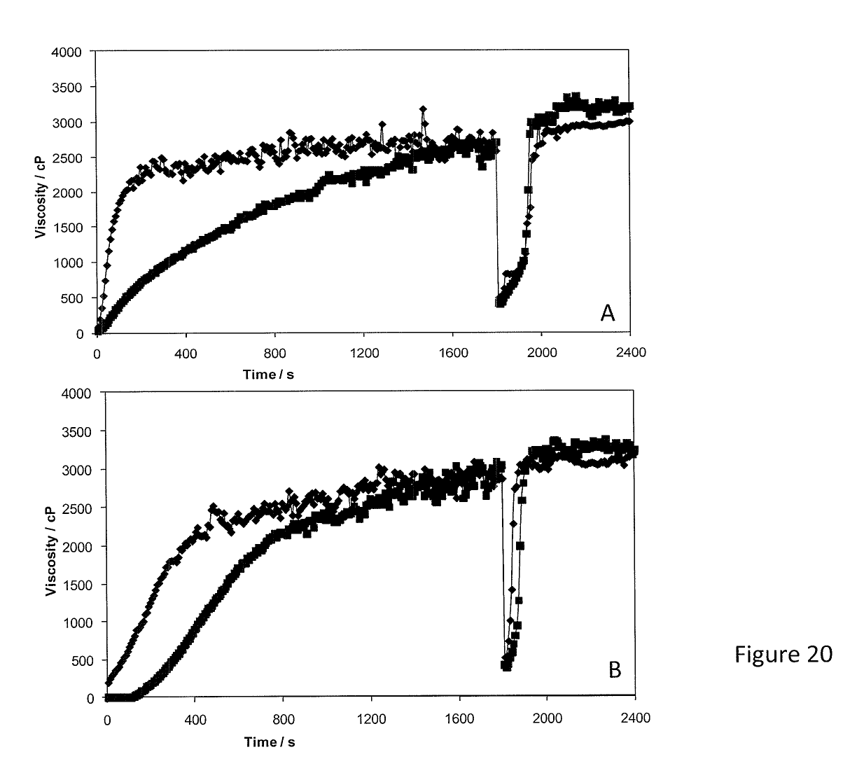

FIG. 20 illustrates the change in viscosity in a Rapid Visco Analyser before (made with water) and after dilution with 0.1M salt solution for a mixture of 10% starch (UT2), 5% oil+2% xanthan gum (♦) and 0.5% xanthan gum (▪). The percentage of dilution (and xanthan type varies) between figures A and B, 20% (supra) 10% (200) respectively.

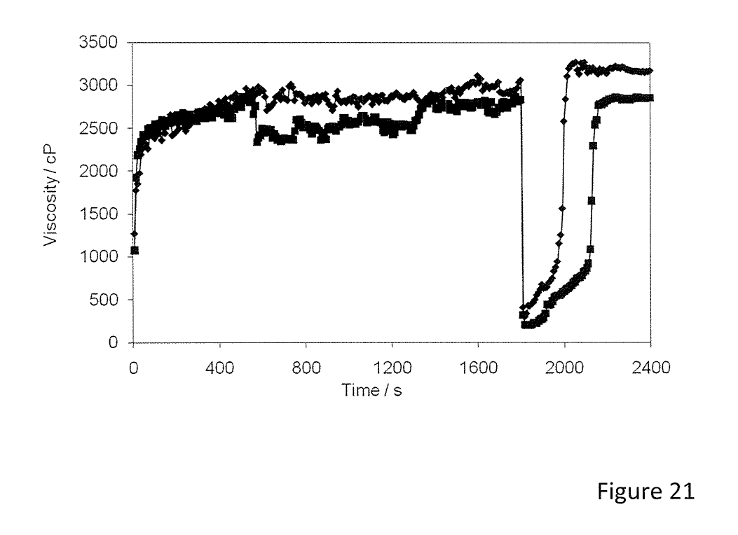

FIG. 21 illustrates the change in viscosity in a Rapid Visco Analyser before (made with water) and after 20% dilution with 0.1M salt solution for a mixture of 10% starch (UT2), 1% oil+2% xanthan gum (♦) and 0.5% xanthan gum (▪).

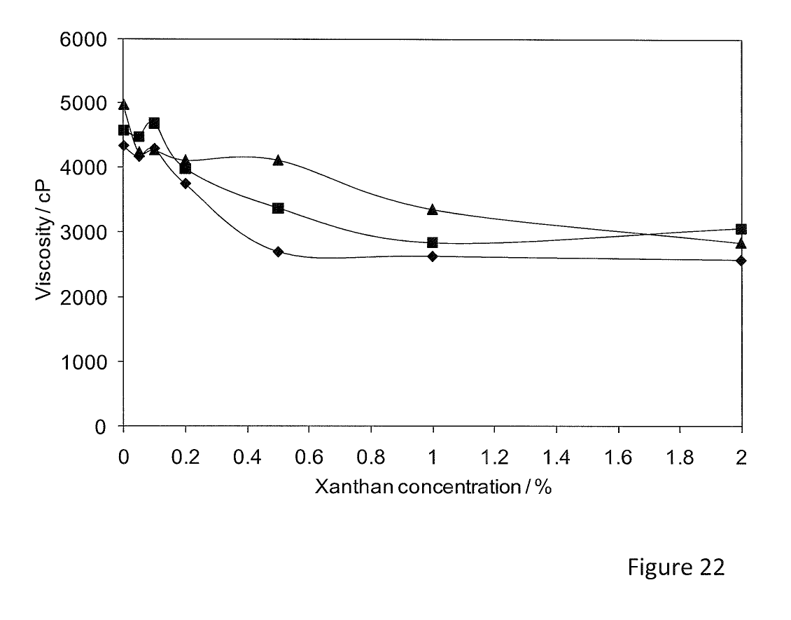

FIG. 22 illustrates final viscosity profiles (after 30 mins of mixing in the Rapid Visco Analyser) as a function of increase in xanthan gum (supra) concentration+5% oil (♦), 1% oil (▪), 1% lecithin (▴) with constant starch (UT2) concentration (10%).

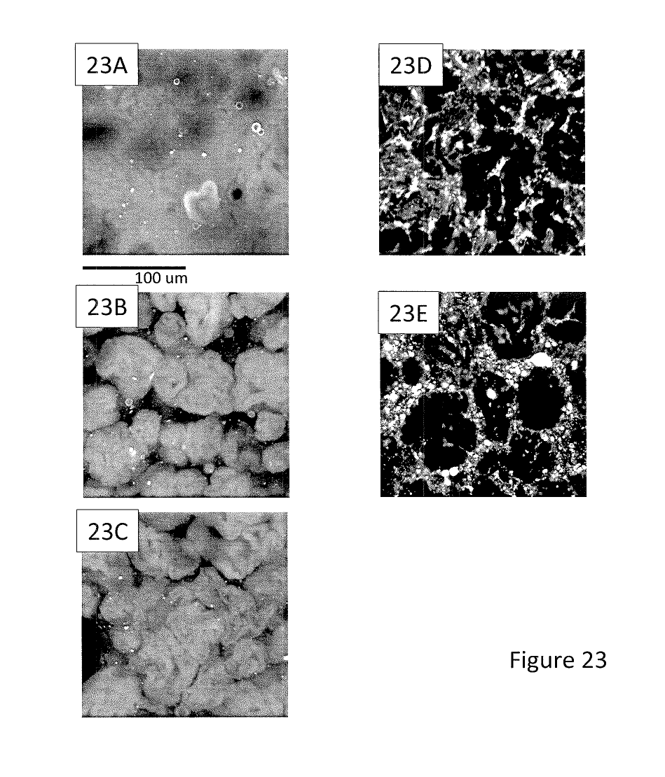

FIG. 23 shows confocal micrographs of 10% starch (UT2)+1% oil (23A)+2% xanthan gum (supra) (23B), upon 20% dilution with 0.1M salt (23C). 10% starch (UT2)+1% lecithin (23D)+2% xanthan gum (supra) (23E)

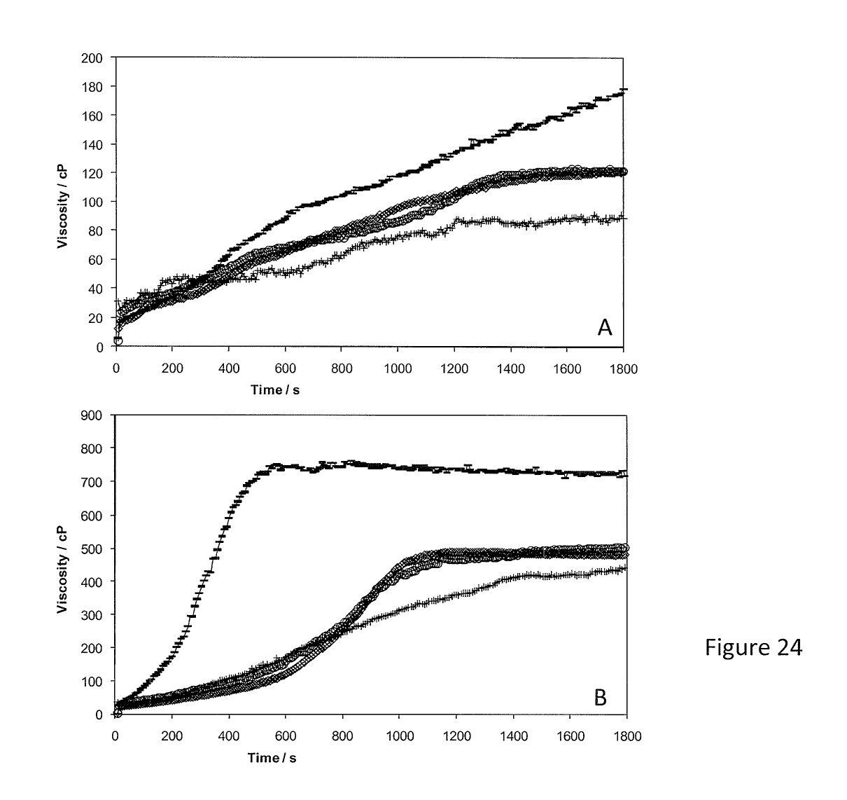

FIG. 24 illustrates the change in viscosity in a Rapid Visco Analyser for control mixtures containing only xanthan (sigma) (+), xanthan (supra) ( [symbol] ), xanthan (200) (⋄) and xanthan (80) (∘)+5% oil. The amount of xanthan varies between figures A and B, 0.5% and 2% respectively.

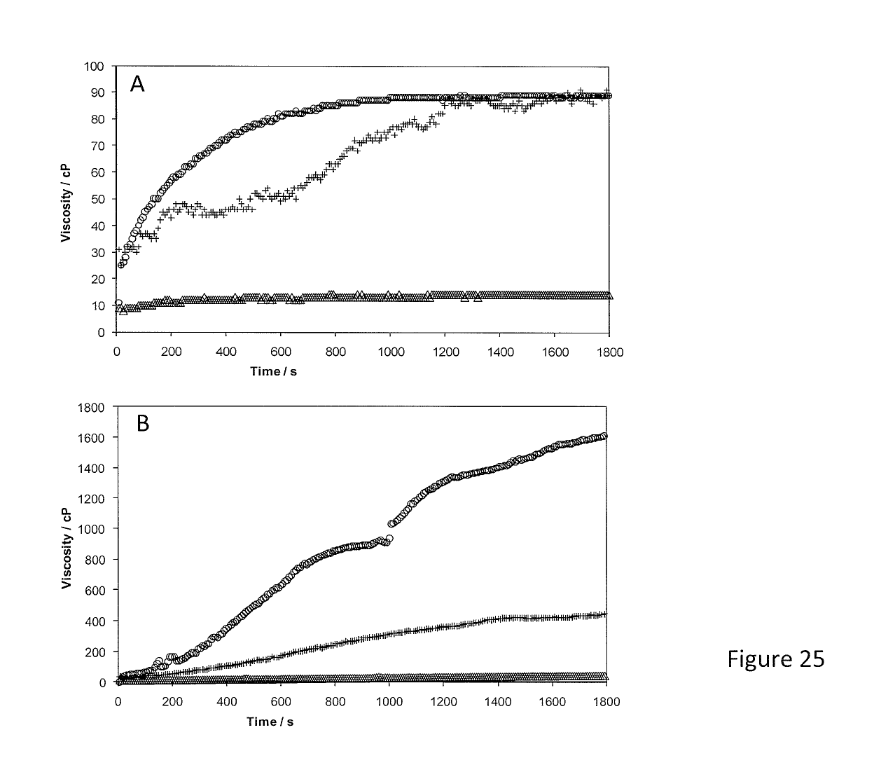

FIG. 25 illustrates the change in viscosity in a Rapid Visco Analyser for control mixtures containing only xanthan gum (sigma) (+), guar gum (Δ), alginate (∘)+5% oil. The amount of hydrocolloid varies between figures A and B, 0.5% and 2% respectively.

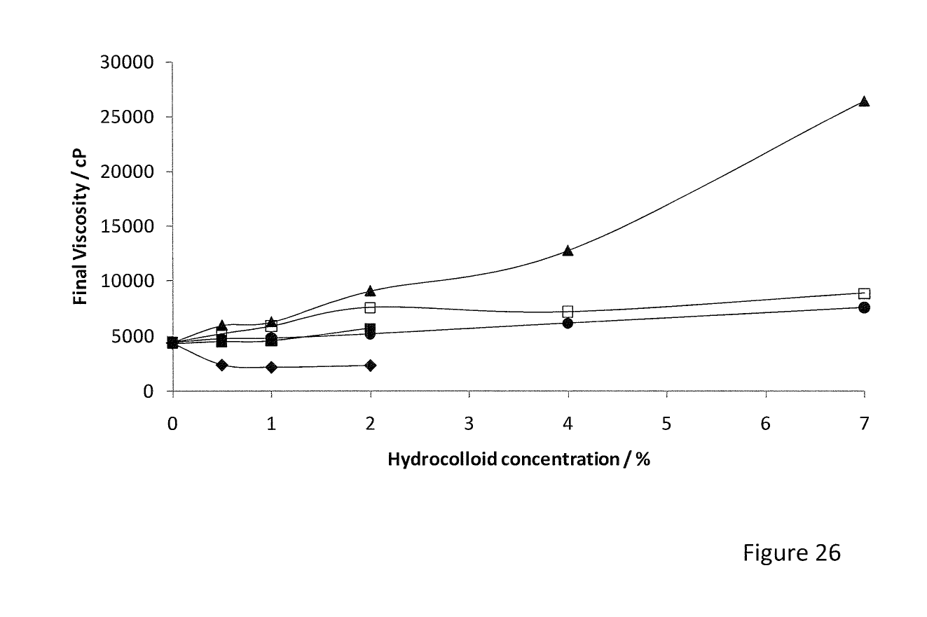

FIG. 26 illustrates final viscosity profiles (after 30 mins of mixing in the Rapid Visco Analyser) as a function of increase in hydrocolloid concentration xanthan sigma gum (♦), guar gum (▪), konjac high MW (▴), konjac low MW () and HPC (□) with constant starch (UT2) concentration (10%).

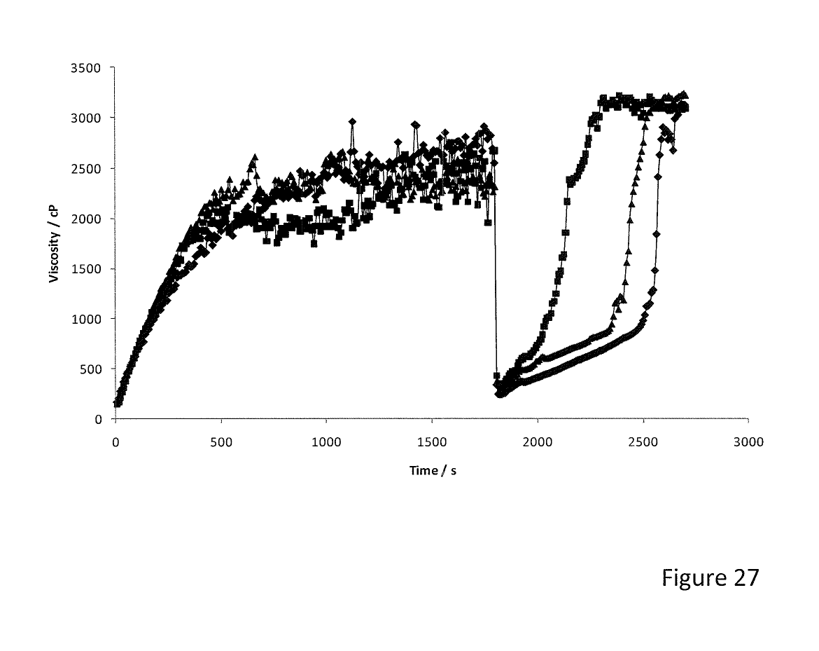

FIG. 27 illustrates the change in viscosity in a Rapid Visco Analyser before (made with water) for a mixture of 10% starch (UT2), 5% oil+2% xanthan gum and after 20% dilution with water (♦), 20% dilution with acid (HCl) (▪) and 20% dilution with 0.1M salt (▴).

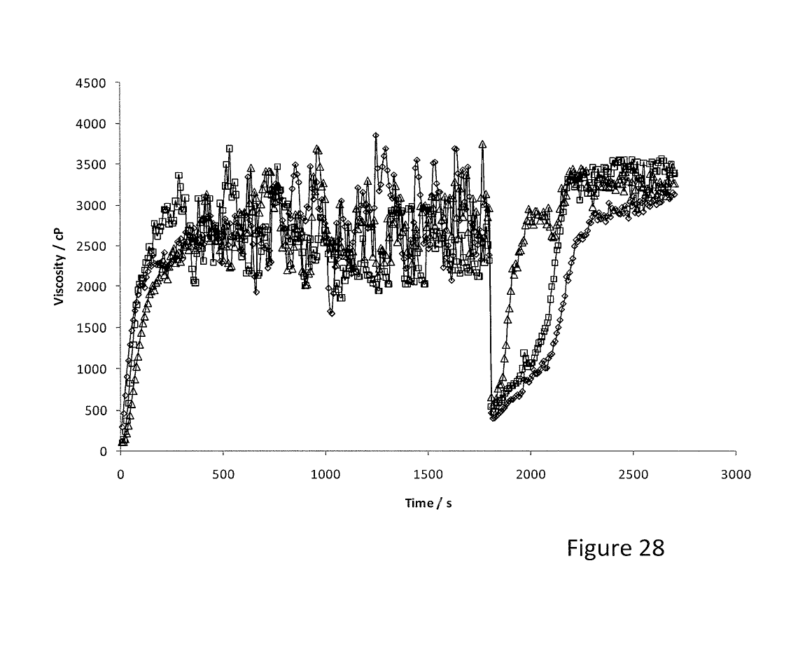

FIG. 28 illustrates the change in viscosity in a Rapid Visco Analyser for a mixture of 10% starch (UT2), 5% oil+2% xanthan gum before dilution made in water (⋄), before dilution made in 0.2M salt (□) and before dilution made in citric buffer pH 4.2 (Δ). All samples were diluted by 20% with water.

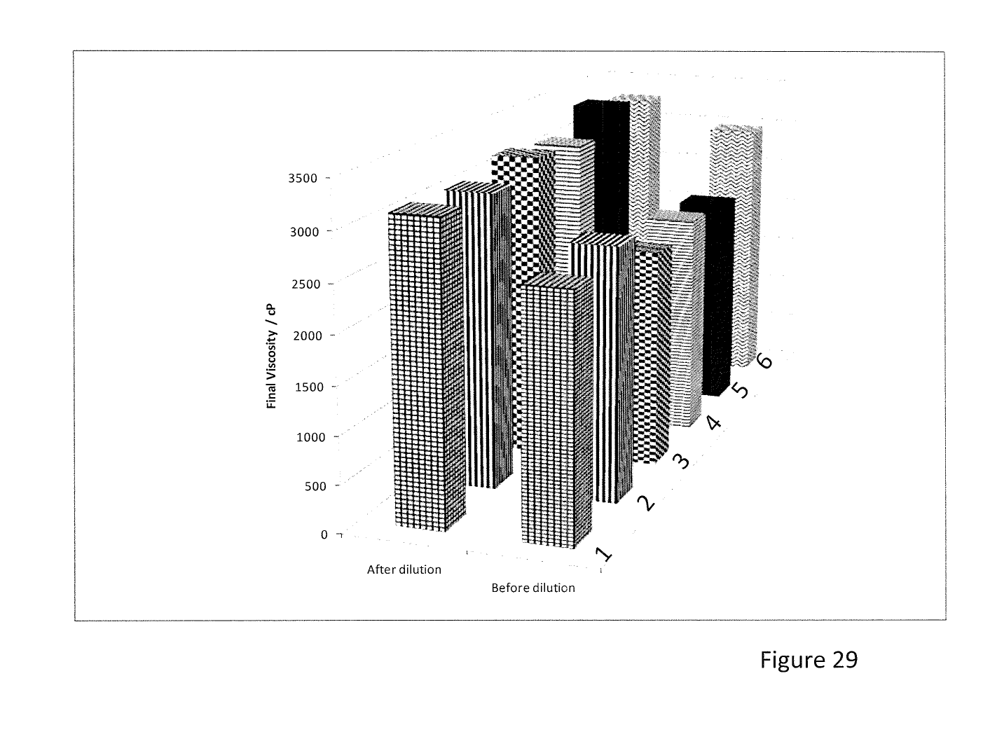

FIG. 29 illustrates final viscosities before (30 mins of mixing in the Rapid Visco Analyser) and after (15 mins of mixing in the Rapid Visco Analyser) dilution where the initial solution within which samples were prepared was water and diluted by 20% with varying solutions water (1), acid (HCl) (2), 0.1 M salt solution (3). Where the initial solution used to make sample varied was 0.1 M salt solution (4), 0.2 M salt solution (5) and citric buffer pH 4.2 (6) diluted by 20% with water. Present in the initial mixture was 10% starch (UT2)+2% xanthan gum and 5% oil.

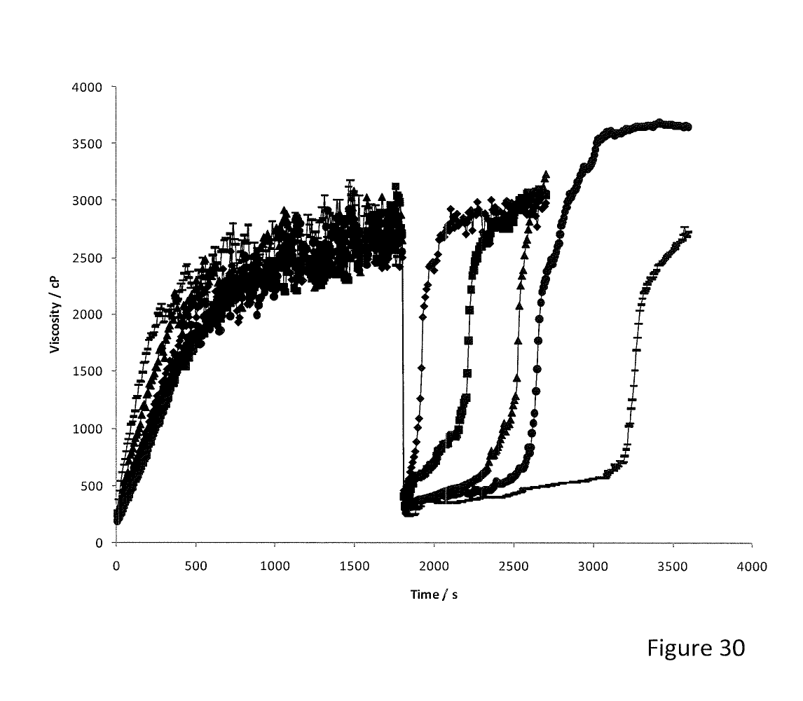

FIG. 30 illustrates the change in viscosity in a Rapid Visco Analyser for a mixture of 10% starch (UT2)+5% oil+2% xanthan gum before (made with water) and after dilution (with water) by 10% (♦), by 20% (▪), by 30% (▴), by 40% () and 50% ( [symbol] )

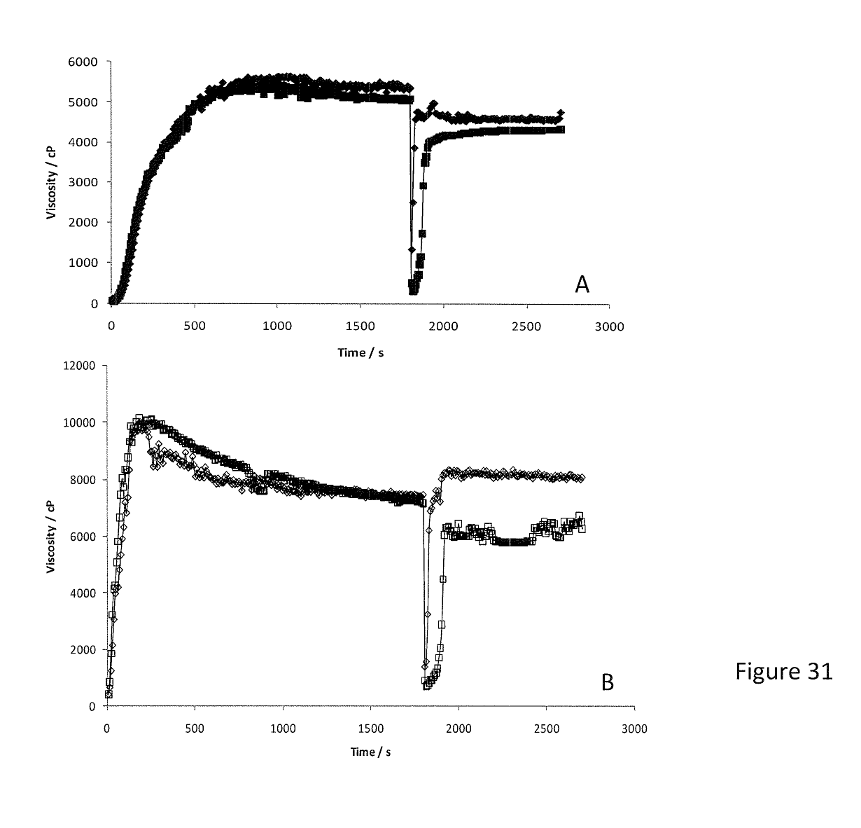

FIG. 31 illustrates the change in viscosity in a Rapid Visco Analyser for a mixture of 10% starch (UT2)+5% oil+2% konjac high MW(A) before (made with water) and after dilution (with water) by 10% (♦) and by 20% (▪). 10% starch (UT2)+5% oil+2% konjac low MW(B) before (made with water) and after dilution (with water) by 10% (⋄)and by 20% (□).

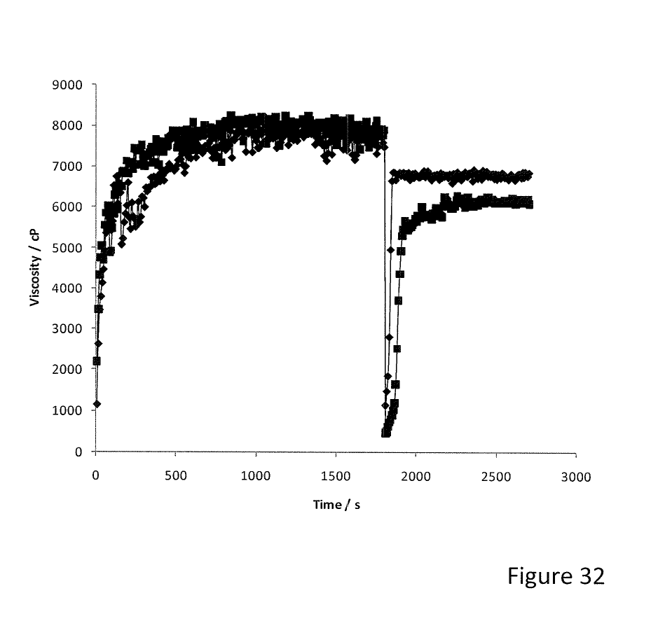

FIG. 32 illustrates the change in viscosity in a Rapid Visco Analyser for a mixture of 10% starch (UT2)+5% oil+2% HPC before (made with water) and after dilution (with water) by 10% (♦) and by 20% (▪).

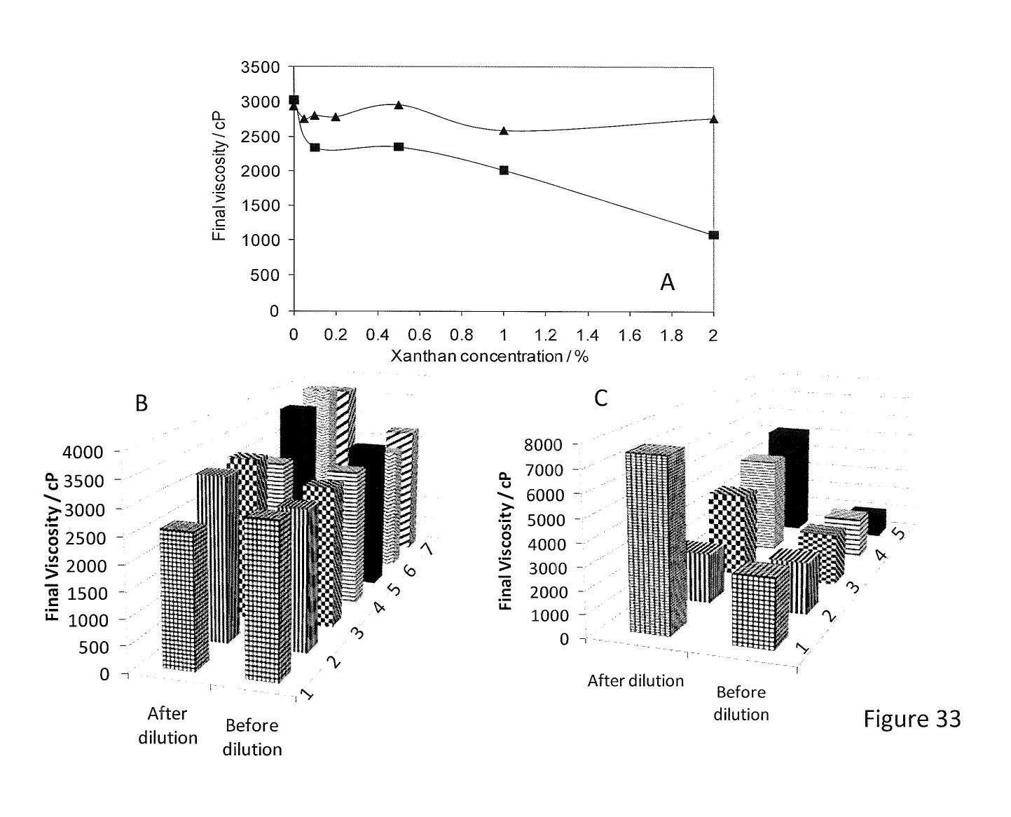

FIG. 33A illustrates final viscosity profiles as a function of increase in xanthan gum concentration, 5% oil+10% hydraxan (250)(▴)and 10% hydraxan (125)(▪). FIG. 33B illustrates the final viscosities before (30 mins of mixing in the Rapid Visco Analyser) and after (15 mins of mixing in the Rapid Visco Analyser) 20% dilution (with water) where the xanthan gum concentration was 0% (1), 0.05% (2), 0.1% (3), 0.2% (4), 0.5% (5), 1% (6) and 2% (7). Also present in the initial mixture was 10% hydraxan(250) and 5% oil. FIG. 33C illustrates the final viscosities before and after 20% dilution (with water) where the xanthan gum concentration was 0% (1), 0.1% (2), 0.5% (3), 1% (4) and 2% (5). Also present in the initial mixture was 10% hydraxan(125) and 5% oil.

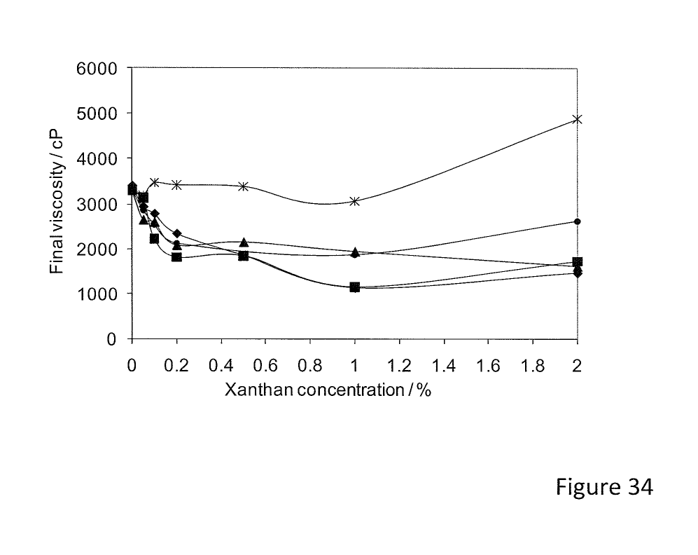

FIG. 34 illustrates final viscosity profiles (after 30 mins of mixing in the Rapid Visco Analyser) as a function of increasing concentration varying types of xanthan gum and guar gum-xanthan gum (sigma) () xanthan gum (supra) (▴), xanthan gum (200) (▪), xanthan gum (80)(♦)and guar gum (*) with constant citrus fibre and oil concentration of 8% and 5% respectively.

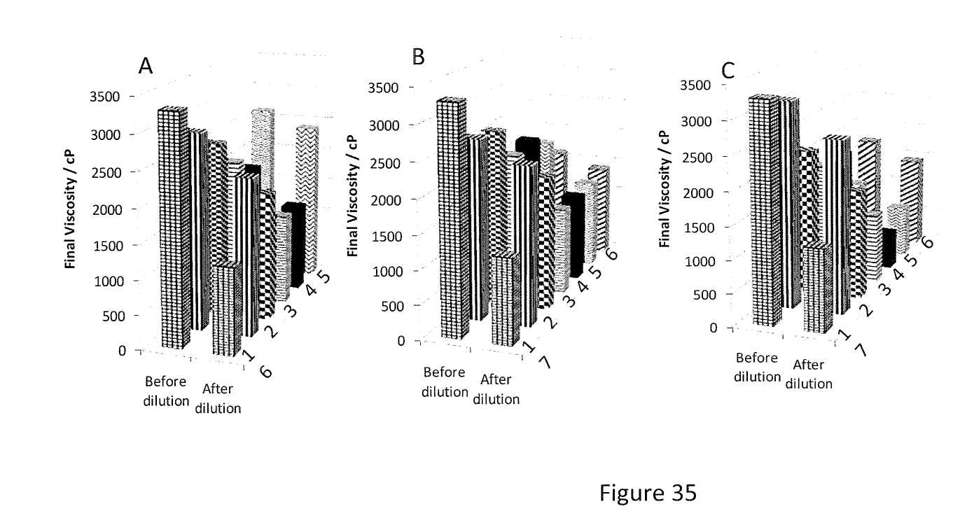

FIG. 35 illustrates final viscosities before (30 mins of mixing in the Rapid Visco Analyser) and after (15 mins of mixing in the Rapid Visco Analyser) 20% dilution (with water) where the xanthan gum (sigma) (A) concentration was 0% (1), 0.05% (2), 0.1% (3), 0.2% (4), 1% (5) and 2% (6). The xanthan gum (supra and 200 figures (B) and (C) respectively) concentration present at 0% (1), 0.05% (2), 0.1% (3), 0.2% (4), 0.5% (5), 1% (6) and 2% (7). All samples contained a constant citrus fibre and oil concentration of 8% and 5% respectively.

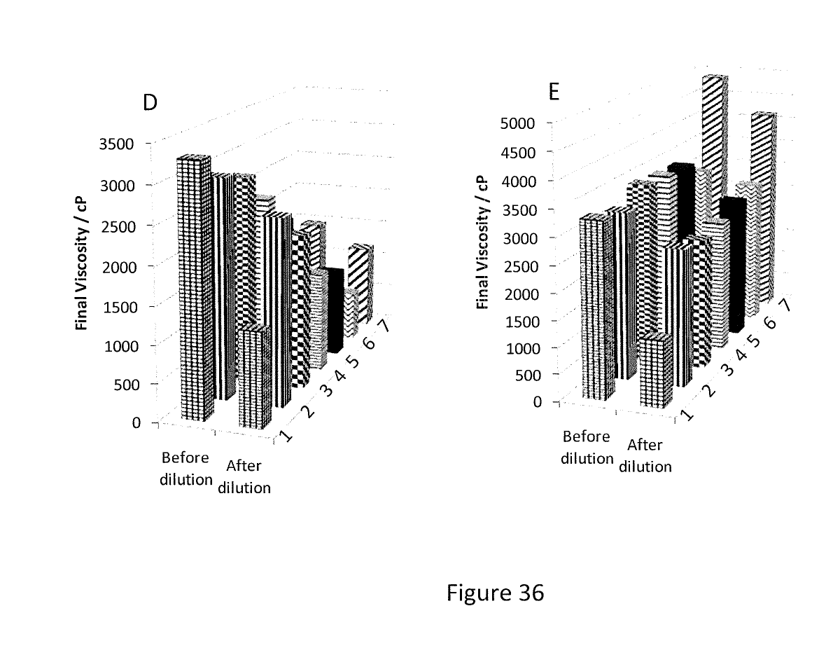

FIG. 36 illustrates final viscosities before(30 mins of mixing in the Rapid Visco Analyser) and after (15 mins of mixing in the Rapid Visco Analyser) 20% dilution (with water) where the xanthan gum (80) (D) and guar gum (E) concentration was 0% (1), 0.05% (2), 0.1% (3), 0.2% (4), 0.5% (5), 1% (6) and 2% (7). All samples contained a constant citrus fibre and oil concentration of 8% and 5% respectively.

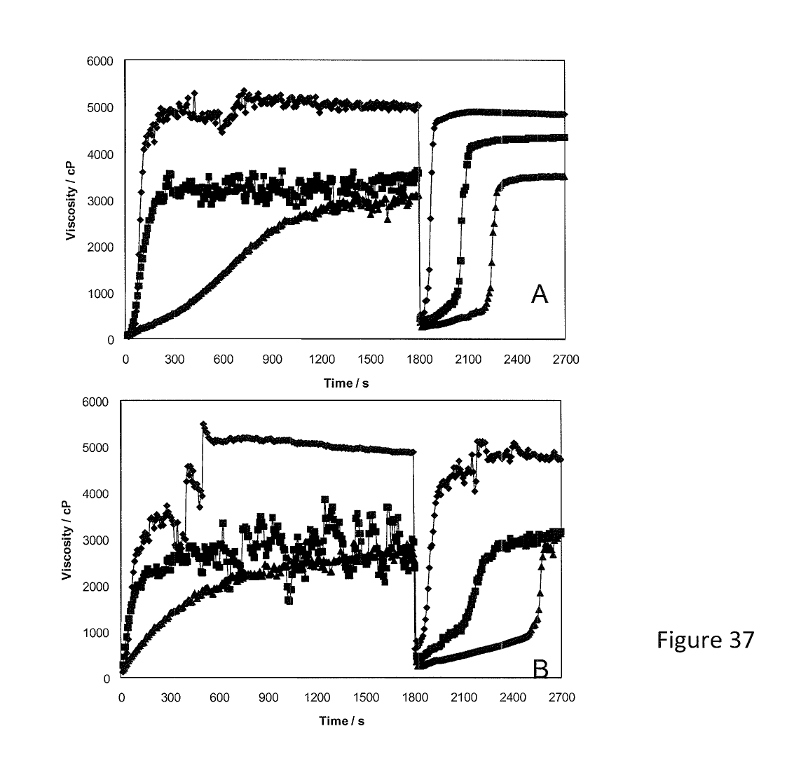

FIG. 37 illustrates the change in viscosity in a Rapid Visco Analyser for a mixture of 10% starch (UT2), 5% oil+xanthan gum before dilution made in 0.01M salt (▴), 0.1M salt (▪) and 1M salt (♦). All samples were diluted by 20% with water. The amount of xanthan gum varies between figures A and B, 0.5% and 2% respectively.

Initially the effect of different concentrations of xanthan gum (and compared to other hydrocolloids: namely, uncharged guar gum and negatively charged alginate) and starch (as a swellable particulate) was studied, this allowed the competitive nature of hydration to be monitored. Once an unexpected effect of viscosity reduction at the higher starch concentrations in the presence of xanthan gum (but not the other hydrocolloids) was established, studies focussed on understanding (investigating the effects of salt, point of addition), exploiting (through dilution studies) and checking formulation white space.

Materials

Starches from two different sources were used as the swellable particulates, firstly a cross linked waxy maize (CLWM) starch and secondly a cross linked potato starch (VA70). Oats (commercially available) were also studied as swellable particulates. Citrus fibre (Citrus fibre N, Herbacel AQ plus) was supplied by Herbafood Ingredients GmbH (Germany). Konjac was supplied by Shimizu Chemical Corporation (Japan) where high molecular weight konjac has product name Propol RS and low molecular weight konjac has product name Reolex LM. Klucel hydroxypropyl cellulose (HPC) was sourced from Hercules Incorporated (Wilminton Del. 19894-007, USA).

Three different types of hydrocolloids were used, xanthan gum, guar gum and alginate. In total four different types of xanthan gum were used, one sourced from sigma biological source Xanthomonas campestris CAS-11138-66-2. The other three selected were all food grade xanthan gums from Danisco (Brabrand, Denmark) and varied in mesh size these were GRINDSTED® Xanthan Clear 200, GRINDSTED® Xanthan Clear 80 and GRINDSTED® Xanthan Clear supra. Guar gum (MEYPRODOR 30) was also supplied by Danisco and had a molecular weight of 423 kDa. The Alginate used was PROTANAL HF12ORB with a M:G ratio of 0.55:0.45, this was supplied by FMCBioPolymer (Cork, Ireland).

Lecithin (Bolec MT) was obtained from Loders Croklaan (Zwijndrecht, The Neatherlands). Sunflower oil was purchased commercially.

All substances were used as supplied without further modification unless stated other wise.

Methods

Viscosity Measurements

Viscosity development and therefore sample hydration was measured using the Rapid Visco Analyzer (RVA) (Newport Scientific, Australia). Viscosity was measured as a function of constant temperature (25° C.) and shear rate (180 rpm) for a total period of 30 minutes unless stated otherwise.

Sample Preparation

A range in concentrations of starch and hydrocolloid were used, this ranged from 0-10% (w/w) and 0-2% (w/w) for starch and hydrocolloid respectively in the final mixture. The concentration of sunflower oil was kept constant at 5% unless stated otherwise. For the RVA measurements, the starch, hydrocolloid and oil were all initially mixed together, to this, the required amount of water was added and placed directly into the RVA without any delay and the viscosity development measured. For measurements performed in the presence of salt three different sodium chloride concentrations were used, 0.01 M, 0.1 M and 1 M.

Confocal Microscopy

Where images are presented these were taken using a Nikon Eclipse Ti inverted confocal microscope, Supplier: Nikon UK Ltd., Kingston upon Thames. The equipment comprises Lasers: Argon Ion 488 nm, Green Helium-Neon 543 nm, Blue diode 405nm, and is fitted with C1 detector unit (3 PMT), a C1 transmitter detector unit (transmitted light), and the data collected and analysed with EZ-C1 Control Software.

Results and Discussion

The effect of different hydrocolloids and of different starch types on the final viscosity of a composition was studied, and the structure of starch was considered.

Starch Hydration in the Presence of Xanthan Gum

Initial viscosity development was measured in the presence of a cold water swelling cross linked waxy maize starch (UT2) at 10% and different xanthan gum types at two different concentrations 0.5% and 2%. FIG. 1 shows the viscosity profiles obtained after 30 minutes of mixing. FIG. 1A shows the profiles with 10% UT2 and 0.5% xanthan gum and FIG. 1B shows viscosity development with 10% UT2 and 2% xanthan gum. Also overlaid in both of these figures are the controls for starch alone and the controls for xanthan gum alone and other hydrocolloids are presented in FIGS. 24 and 25 respectively.

As seen in FIG. 1, the viscosity increase for 10% starch alone is rapid and reaches equilibrium by 200 s. The final viscosity achieved was 4422 cP. The viscosity development shown by considering the different xanthan gum types alone, at 0.5% the viscosity appears the same and is much lower than starch reaching a final viscosity of ˜122 cP. As expected, the final viscosity reached with 2% xanthan gum (FIG. 24B) is higher than 0.5% xanthan gum (FIG. 24A). The viscosity development with 2% xanthan gum alone also shows a difference in the rate of increase when the different sources of xanthan gum are compared (FIG. 24B). Xanthan supra increases in viscosity more rapidly and reaches a higher equilibrium value (718 cP) compared with the other three types (all ˜500 cP) (FIG. 24B). This would fit with the property type of this xanthan gum which is said to have very high cold water dispersability.

The viscosity increase observed in the presence of both starch and xanthan gum is different to both starch alone and xanthan gum alone. The rate at which the viscosity increases is slower when compared to starch alone and reaches a lower final viscosity value ˜3000 cP. In the presence of both 0.5% and 2% xanthan supra the initial rate at which the viscosity increases is faster compared with the other xanthan gum types (sigma, 200 and 80).

This reduction in final viscosity would suggest that the presence of xanthan gum is preventing the starch from hydrating completely as the final viscosity value reached is lower than that for starch alone. Evidence for the competing hydration between the starch and xanthan gum can be seen from the initial rates of hydration. If starch was dominating the hydration then perhaps a faster increase in viscosity would have been observed. This is further supported by the confocal micrographs presented in FIG. 2 where the difference in starch swelling is observed with and without xanthan gum. In the presence of 2% xanthan gum it appears to show that the starch has not hydrated fully.

The starch type was changed to UT4, which is also derived from waxy maize but has been modified differently to UT2 to give different textural properties when dispersed. The viscosity development was then measured in the presence of xanthan gum and the resulting data is displayed in FIG. 3. Like UT2, UT4 rapidly increases in viscosity upon hydration which is due to the fast swelling of the starch. The equilibrium viscosity reached after 30 minutes was 5200 cP which is higher than that reached by UT2.

In the presence of 0.5% and 2% xanthan gum and 10% UT4 (FIG. 3A and 3B respectively), the rate of change and final viscosity observed was similar to that obtained in the presence of UT2 (˜3000 cP). This would suggest that the presence of xanthan gum is delaying the hydration of UT4 thereby not reaching the final viscosity of UT4 alone.

A potato starch (VA70) was the final starch sample studied and the viscosity development with xanthan gum is presented in FIG. 4. The viscosity profile for VA70 alone shows a very rapid increase on addition of water reaching equilibrium much quicker and to a higher viscosity of 5581 cP than UT2 and UT4. When 0.5% xanthan gum is introduced, the equilibrium viscosity is reduced (˜3700 cP) compared to VA70 alone. When the xanthan gum concentration was increased to 2% (FIG. 4B) there is an observation of a further drop in equilibrium viscosity to ˜2700 cP.

In mixtures of VA70 and xanthan gum (regardless of source/type) there appears to be no difference in the viscosity profile and all reach a similar end point. It is only in the presence of VA70 that a clear difference in the final viscosity with the different xanthan gum concentrations is observed, with the lower concentrations providing slightly higher final viscosity values (cf. ˜3500 versus ˜2500 cP).

The viscosity development was also measured in the presence of two other hydrocolloids, uncharged guar gum and alginate which, like xanthan gum, is anionic. The guar gum and alginate viscosity profiles are overlaid with one xanthan gum (sigma) to enable easier comparisons to be made between the hydrocolloids (FIG. 5).

In general, the final viscosity achieved in the presence of guar gum and alginate was much higher than that reached in the presence of xanthan gum or in some cases starch alone. FIG. 5 shows the viscosity development for guar gum and alginate with UT2. In the presence of 0.5% guar gum (FIG. 5A) the viscosity increase is rapid though not as quick as starch alone reaching a final viscosity above UT2 at ˜5300 cP. The viscosity profile in the presence of 0.5% alginate shows a delay before the viscosity begins to increase, though the final viscosity reached was above that in the presence of guar gum and UT2 alone at ˜6000 cP. The final viscosities reached in the presence of both 0.5% guar gum and alginate was much higher than the viscosity achieved in the presence of xanthan gum. The final viscosity of guar gum and alginate containing systems was only slightly enhanced when the hydrocolloid concentration was increased to 2% (FIG. 5B) reaching ˜5500 cP and ˜6150 cP, respectively. There was however a difference in the rate of increase in viscosity in the presence of 2% alginate where the rate is increased and the overall profile mirrors that of guar gum. Guar gum and alginate controls at 0.5% (FIG. 25A) show no significant viscosity development. At 2% there is an increase in viscosity where alginate reaches ˜1600 cP but no significant change in viscosity for guar gum (FIG. 25B).

In the presence of UT4, the final viscosity reached in the presence of 0.5% and 2% guar gum and alginate was higher than that for UT4 alone (FIG. 6). Similar to the results obtained for UT2 and 0.5% alginate, there was an initial delay before the viscosity began to increase but reaches a final viscosity of ˜6150 cP. This delay in viscosity increase was not observed in the presence of guar gum which reached a final viscosity of ˜6150 cP. With an increase in the concentration of guar gum and alginate to 2% the final viscosity increased to ˜6700 and ˜6250 cP, respectively. Unlike in the presence of UT2 where the rate of change in viscosity in the presence of guar gum and alginate was the same with UT4 they both show more of a gradual increase in viscosity. Both the viscosity profiles and final viscosities reached in the presence of guar gum and alginate showed a striking difference compared to that of xanthan gum.

Presented in FIG. 7 are the viscosity profiles of guar gum and alginate in the presence of potato starch VA70. When the hydrocolloid concentration was 0.5% (FIG. 7A) the final viscosity reached was just below the final viscosity of VA70 alone. However, an increase in final viscosity was observed when the concentration of hydrocolloid was 2% (FIG. 7B), a final viscosity of ˜7000 cP for guar gum and ˜6500 cP for alginate was observed. In both FIGS. 7A and 7B the final viscosity observed is higher than in the presence of xanthan gum.

From observing the viscosity profiles of starch in the presence of three different hydrocolloids and at two different concentrations it is clear that xanthan gum behaves in a unique way compared to guar gum and alginate. The results show that the viscosity in the presence of xanthan gum is always much lower than in the presence of the other two hydrocolloids. In order to determine at which concentration of xanthan gum a drop in final viscosity is observed, measurements were made with reduced xanthan gum concentrations and are discussed below.

Varying Hydrocolloid Concentration

Xanthan gum concentration was varied from 0.05% to 2% with the starch concentration being kept constant at 10%. Presented in FIG. 8 are the viscosity profiles after 30 minutes in the presence of 10% UT2 and three different xanthan gum types, sigma, xanthan supra and xanthan 200. What is observed upon varying the xanthan gum concentration is that the final viscosity is generally maintained i.e. similar to UT2 alone (˜4300 cP) when the xanthan gum concentration was 0.05 and 0.1%. The final viscosity begins to drop below that of UT2 alone when the xanthan gum concentration was 0.2% and plateau final viscosities was observed between 0.5 and 2% xanthan gum.

Given that both the starch and xanthan gum are hydrocolloids known for their thickening properties this drop in final viscosity observed upon increasing xanthan gum concentration is quite unique. When the same viscosity development is observed in the presence of guar gum there is an increase in final viscosity as shown in FIG. 9. A tentative explanation for the observed decrease in viscosity takes two forms:
(i) the xanthan gum forms an anisotropic solution, with a concentrated xanthan gum phase, as it hydrates into a liquid crystalline phase, with a subsequent decrease in viscosity,
(ii) the hydration of xanthan gum prevents full hydration and swelling of the starch granules, imparting a decrease in particle dispersed viscosity,

with the potential for both of these phenomena taking place. Indeed, the phase concentration of xanthan gum when combined with low molecular weight polyelectolytes at high concentration is thought to originate from the first suggestion.

Xanthan Gum Salt Effect

Xanthan gum properties are known to have a salt dependency to its polyelectrolyte nature, where the introduction of salt would effectively lead to charge screening. In the absence of salt electrostatic repulsion aids the dispersion of the xanthan gum, resulting in an increased rate of viscosity increase. Salt and xanthan gum concentration determine the final solution viscosity as both have implications on the weak-gel behaviour that a xanthan gum solution exhibits. The transition from isotropic to anisotropic solutions is also known to be salt dependant, with a higher concentration of xanthan gum being required at higher salt levels.

To determine the effect of salt on the final viscosity, the viscosity profiles were repeated by replacing the water with a sodium chloride solution. Three different salt levels were used 0.01M, 0.1M and 1M; the resulting profiles for 10% UT2 with varying xanthan gum (sigma) concentration are shown in FIG. 10. In the presence of low salt (0.01M) the final viscosity curve mirrors the profile shown in the presence of water with the small difference being that the final viscosity observed in the presence of 0.2-2% xanthan gum is slightly higher. When the salt level was increased to 0.1M, the xanthan gum concentration has to reach 1% before a marked drop in final viscosity is observed, this is due to the charge screening effects of the salt whereby a higher xanthan gum concentration is required. Finally, when the highest (1M) salt level is used no significant change in final viscosity is observed. Although not presented, similar trends are also observed with the other xanthan gum types. The presence of salt enables the starch to swell unhindered by the xanthan gum, especially at higher salt concentrations, this is supported further by confocal images presented in FIG. 11 and FIG. 18.

Upon the addition of salt, the electrostatic repulsion of the polyelectrolyte xanthan gum is charge screened and therefore it will not hydrate as quickly. The consequence of this would be to enable a better hydration and swelling of the starch granules (as seen in the confocal micrographs). An increase in salt also would need more xanthan gum to be present if it were to be phase concentrated into its liquid crystalline phase, which in turn would promote an increase in viscosity.

Dilution

The presence of xanthan gum at 0.2% and greater has shown reduced starch swelling and the addition of salt (depending on concentration) has shown that the swelling potential of xanthan gum is reduced thus allowing starch to hydrate leading to higher final viscosities. The hypothesis addressed in this section is that of dilution: does the starch hydrate further and therefore swell upon the addition of more water, or does the xanthan gum, which might be in the liquid crystalline state, hydrate leading to an increase in viscosity.

Dilution was performed on all samples with xanthan gum, guar gum and alginate, the samples were diluted by 10% (1 part composition (for example 20 g of composition) to 0.1 part diluent (for example 2 g water)) and 20% (1 part composition (for example 20 g of composition) to 0.2 part diluent (for example 4 g of water)) with water and the viscosity measured for 15 minutes. The final viscosity upon dilution is dependent upon the concentration of hydrocolloid and the percentage by which the sample is diluted. Table 1, below summarises the general findings upon dilution.

TABLE 1

| | | Viscosity change | |
|---|---|---|---|
| Starch | [Hydrocolloid] | upon dilution | Figure numbers |
| UT2 & UT4 | 0.5% & 2% | 10% & 20% dilution | Figures 12 and 15 |
| | xanthan | Viscosity increases | |
| UT2 & UT4 | 0.5% & 2% | 10% & 20% dilution | Figures 13 and 16 |
| | alginate/guar | Viscosity maintained | |
| | | or decreases | |
| VA70 | 0.5% & 2% | 10% & 20% dilution | Figures 14 and 17 |
| | xanthan | Viscosity decreases | |
| VA70 | 0.5% & 2% | 10% & 20% dilution | |
| | alginate/guar | Viscosity decreases | |

Only samples which contain UT2 and UT4 with xanthan gum showed an increase in viscosity upon dilution, this was not observed with the presence of alginate or guar gum as the hydrocolloid or with VA70 as the starch. Although an increase in viscosity was observed upon dilution with xanthan gum the final viscosity does not reach that of starch alone.

What the dilution results in general show is that the viscosity upon dilution (10% or 20%) will increase in the presence of xanthan gum (0.5% or 2%) with UT2 or UT4. Whereas, the viscosity is either maintained or decreases if guar gum or alginate are present as hydrocolloids. In the presence of VA70, and regardless of hydrocolloid type and concentration (0.5% or 2%), a decrease in viscosity is observed upon 10% and 20% dilution. FIG. 30 shows that viscosity recovery is attained, with time, for dilutions up to 50% (i.e 1 part composition to 0.5 parts diluent).

Dilution with Salt

Dilution with water has shown a viscosity increase in the presence of xanthan gum, samples were then selected to observe the effect on viscosity upon dilution using a salt solution. These measurements were performed to gain an understanding on viscosity change as a function of ionic strength. FIG. 20A shows 10% UT2 with xanthan gum (supra) after 20% dilution with 0.1M NaCl and FIG. 20B shows 10% UT2 xanthan gum (200) after 10% dilution with 0.1M NaCl. The dilution in these examples was measured for 10 minutes

Both FIG. 20A and FIG. 20B show that there is an increase in viscosity upon dilution with salt at both xanthan gum concentrations and dilution percentages.

An increase in ionic strength might be equivalent to a decrease in pH, which happens upon presentation to gastric conditions, and therefore from these results it is reasonable to expect an increase in viscosity in the stomach.

Reduced Oil and Replacing with Lecithin

Oil concentration was reduced from 5% to 1% to observe the effect on viscosity. FIG. 22 shows the resulting final viscosity values obtained with 10% UT2 and varying xanthan gum (supra) concentration. Overlaid with these result is that obtained with 5% oil. The results show that the final viscosity profile is the same as with the presence of 5% oil. Dilution with 20% 0.1M NaCl solution also showed an increase in viscosity (FIG. 21) and the confocal images (FIG. 23) show that the packing/swelling of starch increases with dilution.

Viscosity was also measured by replacing 1% oil with lecithin; here the final viscosity trend observed was slightly different to that observed with 1% oil (FIG. 22). The final viscosity shows a slight decrease upon the addition on xanthan gum (0.005%), the final viscosity remains fairly constant with 0.1-0.5% xanthan gum at ˜4100 cP and decreases further in the presence of 1-20% xanthan gum. In the presence of 2% xanthan gum the final viscosity is at ˜3000 cP which is similar to that seen with 5% oil and 1% oil. Confocal images taken in the presence of 1% lecithin and 1% lecithin with 2% xanthan gum are shown in FIG. 23, they show that the starch has hydrated and is in a dense packed arrangement.

Addition of Powder to Paste

Xanthan gum solutions are generally prepared by dispersing xanthan gum in solution and then heating the solution with constant stirring, the solution is then cooled before use. In order to show that xanthan gum is hydrating in the presence of starch, a xanthan gum solution was made in the conventional way to which starch (UT2) 10% was added as a powder and the viscosity measured with time. FIG. 19 shows that the viscosity instantly increases reaching equilibrium just above 3000 cP. On the same figure is plotted the resulting change in viscosity when xanthan gum (2%, sigma) was introduced to a pre-hydrated starch (UT2) paste. The addition of xanthan gum as a powder shows an instant drop in viscosity. These results indicate that the increase in viscosity upon starch addition is due to the hydration of starch, whereas, the addition of xanthan gum to a starch paste leads to starch deswelling. These observations are further supported by the confocal images. The images show regardless of how the system is made the final conformation/structure is the same.

Varying Hydrocolloid Type

FIGS. 5, 6 and 7 show that when alginate and guar gum in solution are mixed with a swellable particulate the viscosity of the compositions is greater than the swellable particulates alone, whereas the addition of xanthan gum in solution to swellable particulates decreases the viscosity to a level lower than the swellable particulates alone. FIG. 26 shows the viscosity increase upon the addition of two other water soluble hydrocolloids, namely konjac glucomannan and hydroxypropylcellulose (HPC). FIGS. 31 and 32 show that upon dilution of a system containing konjac glucomannan or HPC, respectively, the predominant effect is a decrease in viscosity, as seen for alginate and guar.

Dilution Effects with Different Solvents

FIGS. 27 and 28 support the observation made in FIG. 20, indicating that the recovery of viscosity upon dilution is irrespective of whether the samples are made in water and diluted with salt or acid solution, or made in salt or low pH environments and diluted with water. The experiments are summarised in FIG. 29, where it is clear that a viscosity increase is seen for all samples upon a 20% dilution. FIG. 37 shows that at 1M NaCl the starch has swelled to a greater extent than at lower salt levels, since the viscosity drop upon the addition of xanthan gum is minimal, as already indicated in FIG. 10. Upon a 20% dilution, however, the viscosity is recovered for 1 m NaCl and increased for the lower salt levels.

Varying the Swellable Particulate

Replacing starch with alternative swellable particulates e.g. swellable xanthan gum particulates (hydraxan—an extruded particulate form of xanthan gum) or citrus fibre (FIGS. 33A and 34, respectively) results in compositions which show a decrease in viscosity when xanthan gum is added, compared to the viscosity of the swellable particulate alone. This is a similar trend to that shown in FIG. 8 when starch is the swellable particulate. FIGS. 33B and 33C relate to hydraxan particles of different starting particle size, whereby a 20% dilution in the presence of xanthan gum shows an increase in viscosity. FIGS. 35 and 36 show that upon dilution of a citrus fibre containing composition the viscosity decreases, even though the starting viscosity was lower than the viscosity of citrus fibre alone, indicating a complex interaction between particle swelling and the transition between xanthan gum anisotropic and isotropic phases.

Conclusion

Mixtures of xanthan gum and a swellable particulate show a decrease in viscosity when compared to what might be expected from the viscosities of the two starting components. Furthermore the results presented show that upon addition of further solvent to a composition comprising xanthan gum and a swellable particulate the viscosity may increase, where a decrease in viscosity might be expected.

1. A composition comprising at least about 0.2% w/w xanthan gum and at least about 6% w/w of a swellable particulate.

2. The composition of claim 1 wherein the swellable particulate is swellable at a temperature below about 60° C.

3. The composition of claim 1 wherein the swellable particulate comprises polymer molecules that have been stabilized in the particulate form by either chemical or physical crosslinking.

4. The composition of claim 1 wherein the xanthan gum forms a polymeric solution in water.

5. The composition of claim 1 wherein the swellable particulate comprises one or more of the following: modified or unmodified starch, citrus fibre, particulate xanthan gum, fibrous cellulose, oats, swellable gel particles or surfactant micelles.

6. The composition of claim 5 wherein the swellable particulate comprises starch and wherein the starch derived from potato, maize, tapioca, rice, wheat, cassava, pea or any other suitable material.

7. The composition of claim 1 wherein the composition comprises between about 6% w/w and about 25% w/w of a swellable particulate.

8. The composition of claim 1 wherein the composition comprises between about 0.2% w/w and about 4% w/w xanthan gum.

9. The composition of claim 1 wherein the composition further comprises at least about 5% w/w oil and/or at least about 1% w/w lecithin.

10. The composition of claim 1 wherein the composition further comprises from about 0.01M to about 1M salt.

11. The composition of claim 1 wherein the composition further comprises water.

12. The composition of claim 1 wherein the composition is provided as a paste.

13. The composition of claim 1 wherein the composition has a viscosity lower than would be observed in a composition containing only the swellable particulate.

14. The composition of claim 1 wherein the composition comprises at least about 0.2% w/w xanthan gum and at least about 6% w/w of a modified waxy maize starch or particulate xanthan gum.

15. The composition of claim 1 wherein the composition shows an increase or maintenance in viscosity upon dilution.

16. The composition of claim 1 wherein upon dilution the composition thickens a diluting liquid.

17-20. (canceled)

21. A foodstuff comprising a composition according to claim 1 that upon dilution with a liquid will thicken.

22. A process for thickening a product comprising the step of adding the composition of claim 1 to the product.

23. (canceled)

24. A nutritional, nutraceutical, or pharmaceutical composition comprising the composition of claim 1.

25. The composition of claim 24 for use as a dietary aid and/or for the treatment of obesity.

26-27. (canceled)

28. A composition comprising between about 0.8% and about 60.6% xanthan gum, about 28.6% and about 96.6% of a swellable particulate and about 1.6% and about 44.6% oil or lecithin.

29. (canceled)

30. The process of claim 22, wherein the product is a liquid.

31. The process of claim 22, wherein the product is a food stuff.

32. The process of claim 31, wherein the food stuff contains at least about 6% w/w of a swellable particulate.
